(19) United States
(12) Patent Application Publication
(10) Pub. No.: US 2011/0066187 A1
(43) Pub. Date: Mar. 17, 2011

(54) SPINAL STABILIZATION SYSTEM

(75) Inventors: ZHIBIN FANG, EDEN PRAIRIE, MN (US); BRIAN MARQUARDT, WARSAW, IN (US); KAI ZHANG, WOODBURY, MN (US); JOHN M. DAWSON, CHASKA, MN (US); HUGH D. HESTAD, EDINA, MN (US)

(73) Assignee: ZIMMER SPINE, INC., MINNEAPOLIS, MN (US)

(21) Appl. No.: 12/558,170

(22) Filed: Sep. 11, 2009

(51) Int. Cl. A61B 17/70; A61B 17/86; A61B 17/88

(52) U.S. Cl. 606/254; 606/264; 606/305; 606/279

(57) ABSTRACT

A vertebral fixation system including an elongate rod and a vertebral anchor for securement to a vertebra. The vertebral anchor includes a head portion for receiving a portion of the rod. The elongate rod may be formed of a material having a modulus of elasticity less than or equal to 110 GPa and an ultimate strength greater than 1 GPa. The elongate rod may have a structural bending stiffness in the range of about 500,000 N-mm² to about 2,000,000 N-mm² or about 1,250,000 N-mm². In some instances, the elongate rod may be formed of a beta titanium alloy such as high strength Ti-15Mo-5Zr. In some instances the elongate rod has a diameter in the range of about 3.25 millimeters to about 4.5 millimeters. Various elongate rods including regions for receiving a flexible member along an exterior surface of the elongate rods are also provided.

### TECHNICAL FIELD

The disclosure is directed to a spinal stabilization system for securing to a spinal column. More particularly, the disclosure is directed to spinal stabilization systems including elongate rods having desired stiffness and strength characteristics and/or elongate rods having regions for receiving a flexible member.

### BACKGROUND

Commercially available spinal fixation systems for the lumbar region of the spinal column typically use fixed angle or polyaxial bone screws attached to adjacent vertebrae with a 5.5 or 6.0 millimeter diameter metallic rod extending between adjacent bone screws and secured thereto with a cap screw or other fastening member. Commonly used materials for the rods include stainless steel, commercially pure (CP) titanium, alpha-beta titanium alloy, such as Ti-6Al-4V and Ti-6Al-7Nb, or cobalt-chromium-molybdenum alloy (Co—Cr—Mo). Due to their relatively high rigidity compared to the natural spine, these systems have been referred to as a “rigid system”.

However, it has been found that the use of a rigid system may lead to adverse effects to the spinal column. For instance, it is believed that the high degree of stiffness of rigid systems may relate to increased stress on adjacent discs and facet joints. Over time, these increased stresses may lead to segment hypermobility, facet hypertrophy, osteophyte formation, and stenosis or so called adjacent level disease.

Recently, less rigid polymeric or carbon fiber rod systems have been introduced as an alternative to potentially reduce the stress on adjacent discs and facet joints and the incidences of adjacent level disease. These systems have been referred to as a “flexible system”. Commonly used materials for the polymer rod systems include polyether ether ketone (PEEK), PEEK composites, or other polymer materials. These systems, while more flexible than a rigid system, present their own shortcomings, such as the relatively low ultimate strength of the polymeric materials.

In view of the limitations of systems using these types of spinal rods, there is an ongoing need to provide alternative spinal stabilization systems for stabilization of spinal segments of the spinal column which include spinal rods having desired stiffness and/or strength characteristics and/or elongate rods having regions for receiving a flexible member.

### SUMMARY

The disclosure is directed to several alternative designs, materials and methods of manufacturing medical device structures and assemblies.

Accordingly, one illustrative embodiment is a vertebral stabilization system including an elongate rod and a vertebral anchor for securement to a vertebra. The vertebral anchor includes a head portion for receiving a portion of the rod. The elongate rod may be formed of a material having a modulus of elasticity less than or equal to 110 GPa and an ultimate strength greater than 1 GPa. The elongate rod may have a structural bending stiffness in the range of about 500,000 N-mm² to about 2,000,000 N-mm² or about 1,250,000 N-mm². In some instances, the elongate rod may be formed of a beta titanium alloy such as high strength Ti-15Mo-5Zr. In some instances the elongate rod has a diameter in the range of about 3.25 millimeters to about 4.5 millimeters.

Another illustrative embodiment is a vertebral stabilization system for a spinal column. The system includes a vertebral anchor for securement to a vertebra, an elongate rod, a flexible member, and a securing member to secure the elongate rod and the flexible member to the vertebral anchor. The vertebral anchor includes a head portion having first and second arms extending from a base of the head portion, where the head portion includes a channel defined between the first and second arms extending between a first side and a second side of the head portion. The elongate rod has a first region and a second region, wherein the first region of the elongate rod includes an outer surface having an engagement surface portion. The flexible member has a first region and a second region in which the first region of the flexible member is positionable adjacent the engagement surface portion of the first region of the elongate rod when the first region of the elongate rod and the first region of the flexible member are received in the channel of the head portion of the vertebral anchor. The securing member is configured to engage the first and second arms of the head portion of the vertebral anchor to secure both the elongate rod and the flexible member in the channel of the head portion of the vertebral anchor.

Another illustrative embodiment is a method of stabilizing the spinal column of a patient. The method includes securing first and second vertebral anchors to first and second vertebrae of the spinal column on a first, lateral side of the spinal column, and securing third and fourth vertebral anchors to the first and second vertebrae of the spinal column on a second, contra-lateral side of the spinal column. A first elongate rod is secured to the first and second vertebral anchors on the first, lateral side of the spinal column, and a second elongate rod is secured to the third and fourth vertebral anchors on the second, contra-lateral side of the spinal column. A spinal load is transferred between the first and second vertebrae such that between 17% to 19% of the spinal load is transferred through the posterior elements of the first and second vertebrae.

Yet another illustrative embodiment is a method of stabilizing a lumbar region of a spinal column. The method includes installing a first vertebral anchor on a first lumbar vertebra and a second vertebral anchor on a second lumbar vertebra. An elongate rod, having a diameter of less than 5.5 millimeters, may then be secured between the first vertebral anchor and the second vertebral anchor. In some instances, the elongate rod is formed of a material having a modulus of elasticity less than or equal to 110 GPa and an ultimate strength greater than 1 GPa. In some instances the elongate rod is formed of high strength Ti-15Mo-5Zr and has a diameter in the range of about 3.25 millimeters to about 4.5 millimeters. In some instances, the elongate rod has a structural bending stiffness in the range of about 500,000 N-mm² to about 2,000,000 N-mm².

Still another illustrative embodiment is a vertebral stabilization system including an elongate rod, a vertebral anchor for securement to a vertebra, and a securing member configured for securement of the elongate rod to the vertebral anchor. The elongate rod has a diameter of 4.5 millimeters or less. The vertebral anchor includes a head portion having a first leg, a second leg and a channel extending between the first leg and the second leg for receiving the elongate rod. The securing member includes a first component rotatably coupled to a second component. The first component is configured for engagement with the first and second legs of the head portion of the vertebral anchor, and the second component is configured for engagement with the elongate rod. The elongate rod secured to the head portion of the vertebral anchor with the securing member has a fatigue strength greater than a spinal rod of a diameter of 5.5 millimeters formed of any of stainless steel, commercially pure (CP) titanium, Ti-6Al-4V alpha-beta titanium alloy, Ti-6Al-7Nb alpha-beta titanium alloy, or cobalt-chromium-molybdenum alloy (Co—Cr—Mo) secured to a bone screw with a set screw in direct contact with the spinal rod

The above summary of some example embodiments is not intended to describe each disclosed embodiment or every implementation of the invention.

### BRIEF DESCRIPTION OF THE DRAWINGS

The invention may be more completely understood in consideration of the following detailed description of various embodiments in connection with the accompanying drawings, in which:

FIG. 1 is a perspective view of an exemplary embodiment of a vertebral stabilization system;

FIG. 2 is an exploded view of a pair of vertebral anchors and an elongate connecting member of the vertebral stabilization system of FIG. 1;

FIG. 3 is a perspective cross-sectional view of the securing member of the vertebral stabilization system of FIG. 1;

FIG. 4 is a chart comparing the elastic modulus of these commercially available spinal rod materials;

FIG. 5 is a chart illustrating the relative structural bending stiffness of commercially available spinal rods of various materials as a percentage of structural bending stiffness of a 5.5 millimeter rod formed of Ti-6Al-4V;

FIG. 6 is a chart comparing the ultimate strength of commercially available spinal rod materials;

FIG. 7 is a graph illustrating the percent of load sharing of an axial spine compression load of 5.5 millimeter spinal rods formed of PEEK and Ti-6Al-4V relative to the structural bending stiffness of the spinal rod as a percentage of the structural bending stiffness of a 5.5 millimeter spinal rod formed of Ti-6Al-4V;

FIG. 8 is a chart illustrating the structural bending stiffness of connecting members formed of high strength beta titanium alloy Ti-15Mo-5Zr material and having diameters of 3.25 millimeters, 3.75 millimeters, 4 millimeters, and 4.5 millimeters;

FIG. 9 is a chart comparing the fatigue strength of a 4.25 millimeter diameter rod formed of Ti-15Mo-5Zr to the fatigue strength of a 5.5 millimeter rod formed of CP titanium;

FIGS. 10A and 10B are perspective views of an exemplary transverse connector for use in the vertebral stabilization system of FIG. 1;

FIG. 10C is a perspective longitudinal cross-sectional view of the transverse connector of FIGS. 10A and 10B;

FIG. 11A is a perspective view of an alternative embodiment of a transverse connector for use in the vertebral stabilization system of FIG. 1;

FIG. 11B is a longitudinal cross-sectional view of the transverse connector of FIG. 11A;

FIG. 12 is a perspective view of another exemplary vertebral stabilization system;

FIG. 13 is a perspective view of an elongate rod of the vertebral stabilization system of FIG. 12;

FIG. 14 is a perspective view of an alternative elongate rod of the vertebral stabilization system of FIG. 12;

FIG. 15 is a longitudinal cross-sectional view of the vertebral stabilization system of FIG. 12;

FIG. 16 is a perspective view of another exemplary vertebral stabilization system;

FIG. 17 is an exploded view of the vertebral stabilization system of FIG. 16; and

FIG. 18 is a perspective cross-sectional view of the vertebral stabilization system of FIG. 16.

While the invention is amenable to various modifications and alternative forms, specifics thereof have been shown by way of example in the drawings and will be described in detail. It should be understood, however, that the intention is not to limit aspects of the invention to the particular embodiments described. On the contrary, the intention is to cover all modifications, equivalents, and alternatives falling within the spirit and scope of the invention.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (i.e., having the same function or result). In many instances, the term “about” may be indicative as including numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

Although some suitable dimensions, ranges and/or values pertaining to various components, features and/or specifications are disclosed, one of skill in the art, incited by the present disclosure, would understand desired dimensions, ranges and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The following detailed description should be read with reference to the drawings in which similar elements in different drawings are numbered the same. The detailed description and the drawings, which are not necessarily to scale, depict illustrative embodiments and are not intended to limit the scope of the invention. The illustrative embodiments depicted are intended only as exemplary. Selected features of any illustrative embodiment may be incorporated into an additional embodiment unless clearly stated to the contrary.

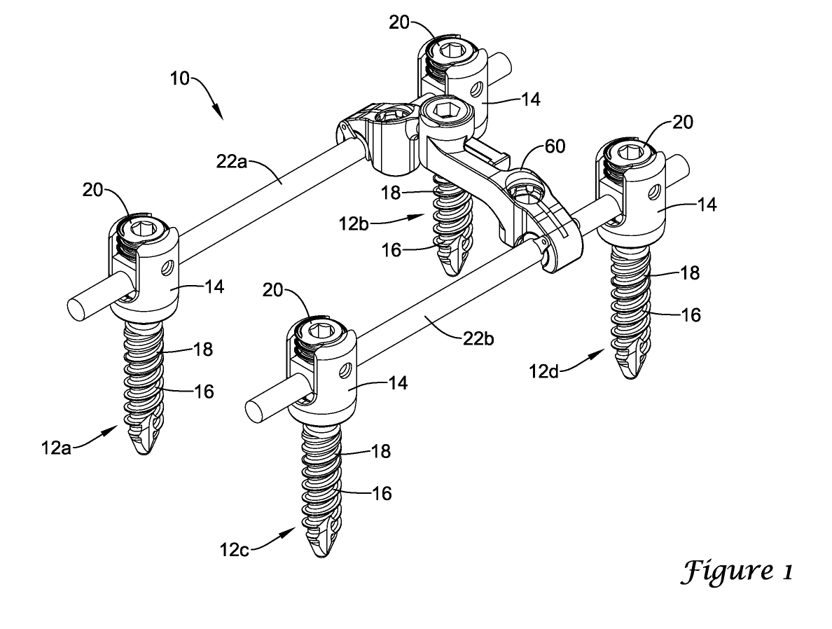

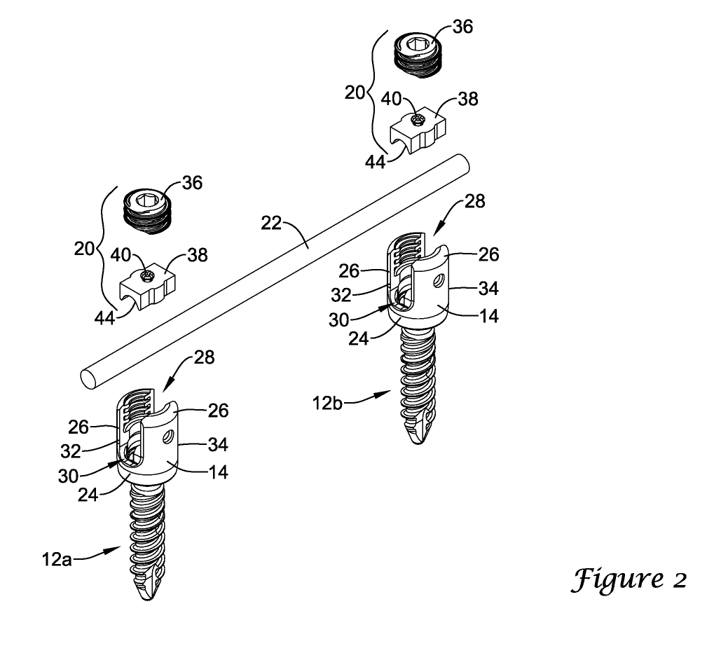

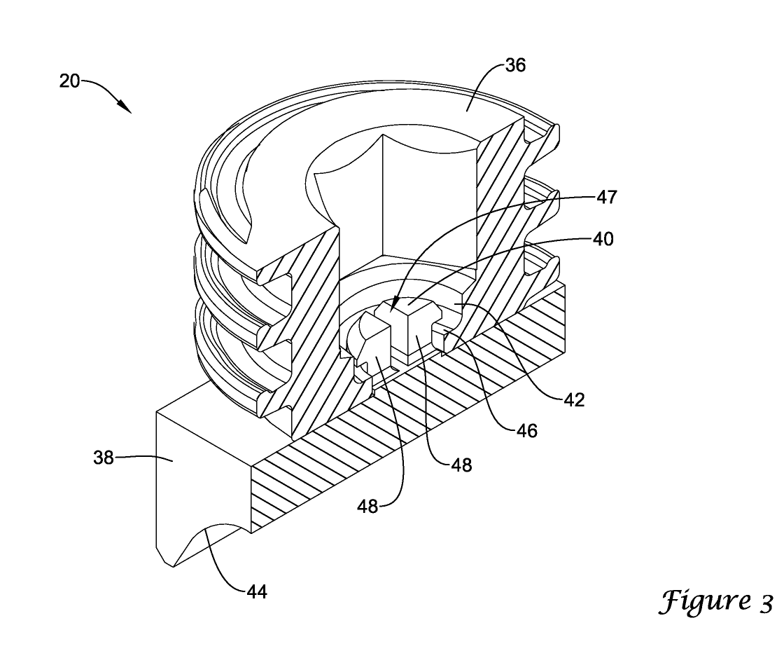

Now referring to the drawings, an exemplary vertebral fixation system 10 for stabilizing a portion of a spinal column, such as one or more spinal segments of a spinal column, is illustrated in FIGS. 1 and 2. As used herein, a spinal segment is intended to refer to two or more vertebrae, the intervertebral disc(s) between the vertebrae and other anatomical elements between the vertebrae. For example, a spinal segment may include first and second adjacent vertebrae and the intervertebral disc located between the first and second vertebrae. The vertebral stabilization system 10 may provide support to the spinal segment and may help preserve the facet joints between adjacent vertebrae by providing facet offloading and/or may stabilize or reverse neural foraminal narrowing of the spinal column.

In some embodiments, the vertebral stabilization system 10 may be used to treat discogenic low back pain, degenerative spinal stenosis, disc herniations, facet syndrome, posterior element instability, adjacent level syndrome associated with spinal fusion, and/or other maladies associated with the spinal column.

The vertebral stabilization system 10 may include one or more or a plurality of vertebral anchors or fasteners 12. Although the vertebral anchors 12 are depicted as threaded vertebral fasteners (e.g., pedicle screws, bone screws), in some embodiments the vertebral anchors 12 may be vertebral hooks (e.g., laminar hooks) or other types of fastening members for attachment to a bony structure such as a vertebra of the spinal column. Each of the vertebral anchors 12 may be configured to be secured to a vertebra of a spinal column. For instance, the first vertebral anchor 12a may be secured to a first vertebra and the second vertebral anchor 12b may be secured to a second vertebra. In a multi-lateral application, the third vertebral anchor 12c may be secured to the first vertebra and the fourth vertebral anchor 12d may be secured to the second vertebra on a contra-lateral side of the sagittal plane. Additional vertebral anchors 12 may be secured to additional vertebrae as desired.

The vertebral anchor 12 may include a head portion 14 and a bone engagement portion 16 extending from the head portion 14. In some embodiments, the bone engagement portion 16 may be a shaft portion 18 of the vertebral anchor 12 extending from the head portion 14 along a longitudinal axis of the vertebral anchor 12. In some embodiments, the vertebral anchor 12 may be a monoaxial screw in which the head portion 14 is stationary relative to the shaft portion 18, and in other embodiments the vertebral anchor 12 may be a polyaxial screw in which the head portion 14 is actuatable (e.g., pivotable) relative to the shaft portion 18. In some embodiments, the shaft portion 18 may be configured to be installed into a bony region of a vertebra of the spinal column. For example, the shaft portion 18 may be installed into a pedicle of a vertebra, or other region of a vertebra. In some embodiments, the shaft portion 18 may be a threaded region having helical threads configured to be screwed into a pedicle of a vertebra, or other bony region of a vertebra.

The head portion 14 may include a base portion 24, from which the shaft portion 18 extends from, and first and second legs 26 extending from the base portion 24 on opposing sides of the head portion 14. The first and second legs 26 may define an opening 28, which may be a threaded opening in some instances, extending into the head portion 14 from an upper extent of the head portion 14 opposite the base portion 24. In embodiments in which the opening 28 is threaded, each of the first and second legs 26 may include a threaded portion for threadedly engaging a threaded portion of a securing member 20. In other embodiments, the first and second legs 26 may include other engagement features for engaging with a securing member positioned in the opening 28 between the first and second legs 26. The head portion 14 may additionally include a channel 30, such as a U-shaped channel, defined between the first and second legs 26. The channel 30 may extend through the head portion 14 from a first side 32 of the head portion 14 to a second side 34 of the head portion 14. The opening 28 may intersect the channel 30.

The vertebral anchor 12 may include a securing member 20 configured to engage the head portion 14 to secure a stabilizing member or connecting member 22 (e.g., elongate rod or flexible cord) to the vertebral anchor 12. For example, the securing member 20 may include a first component rotatably coupled to a second component. For instance, the securing member 20 may include an upper threaded screw 36 rotatably coupled to a lower, insert 38. The threaded screw 36 may be rotated relative to the insert 38 about a pivot axis. The threads of the threaded screw 36 may mate with threads formed in the head portion 14. For example, the threads of the threaded screw 36 may mate with threaded portions of the first and second legs 26 of the head portion 14. In other embodiments, other securing members, such as threaded fasteners, may be used to secure a connecting member 22, such as an elongate rod or flexible member, in the head portion 14 of the vertebral anchor 12.

The threaded screw 36 may be formed of a first, rigid material such as metal, including stainless steel, titanium, titanium alloys or other metal, while the insert 38 may be formed of a different material. For instance, in some cases the insert 38 may be formed of a polymeric material, such as polyether ether ketone (PEEK), carbon fiber reinforced PEEK, ultra high molecular weight polyethylene (UHMWPE), or poly(methyl methacrylate) (PMMA). The insert 38 may be rotatably attached to the threaded screw 36 with a boss 40 that extends into an opening 42 of the threaded screw 36, in some instances. For instance, the boss 40 may include an enlarged diameter portion that extends through the opening 42 and engages a rim 46 of the opening 42. The boss 40 may be sufficiently deflectable or compressible such that the enlarged diameter portion of the boss 40 (which has a diameter or cross sectional distance greater than a diameter of the opening 42) may be urged through the opening 42, but then retained in the opening 42 during usage. In some instances, the boss 40 may include one or more slots 47 dividing the boss 40 into a plurality of prongs 48 for allowing one or more of the prongs 48 of the boss 40 to deflect radially inward toward the pivot axis of the threaded screw 36 in order to allow the boss 40 to be urged through the opening 42. Once inserted into the opening 42, the interaction of the boss 40 with the rim 46 of the opening 42 may retain the insert 38 rotatably coupled to the threaded screw 36.

The insert 38 may include a cylindrically concave lower surface 44 for contacting the cylindrical outer surface of a connecting member 22 when positioned in the head portion 14 of a vertebral anchor 12. The insert 38 may be configured to allow a connecting member 22 having a diameter less than 5.5 millimeters to be secured in the channel 30 of the head portion 14 of a vertebral anchor 12 which is sized to receive a 5.5 millimeter or greater diameter rod (e.g., a vertebral anchor in which the channel of the head portion has a width measured between the first leg and the second leg of 5.5 millimeters or more). For instance, the lower surface 44 may have a radius of curvature approximating the radius of the connecting member 22. For example, the radius of curvature of the lower surface 44 may be about 5.0 millimeters, about 4.5 millimeters, about 4.0 millimeters, about 3.75 millimeters, about 3.5 millimeters, or about 3.25 millimeters in some instances.

Furthermore, the insert 38 may more evenly distribute a securing force on the connecting member 22 in order to prevent notching of the connecting member 22 by a set screw. For instance, the presence of the insert 38 between the threaded screw 36 and the connecting member 22 prevents the threaded screw 36 from directly engaging the connecting member 22. Thus, the insert 38 may provide a buffer to notching, pitting, fretting or galling of the connecting member 22 (which can reduce the fatigue strength of the connecting member 22) from direct contact with a set screw or other threaded fastener, while the threaded screw 36 may still be used to secure the connecting member 22 in the head portion 14 of the vertebral anchor 12. The presence of the insert 38 between the threaded screw 36 and the connecting member 22 may provide a stress gradient between the threaded screw 36 and the connecting member 22, distributing the forces exerted onto the connecting member 22 through tightening the threaded screw 36 in the head portion 14 of the vertebral anchor 12 over a larger portion of the exterior surface of the connecting member 22. Therefore, the inclusion of the insert 38 may eliminate the notch sensitivity of the connecting member 22, thereby increasing the fatigue strength of the connecting member 22 in the vertebral stabilization system 10 such that a connecting member 22 of a smaller diameter than conventional spinal rods (e.g., smaller than 5.5 millimeters) may be used in the vertebral stabilization system 10 while not compromising the fatigue strength of the connecting member 22 and the vertebral stabilization system 10. In many instances, the inclusion of the insert 38 greatly increases the fatigue strength of the connecting member 22 and the vertebral stabilization system 10. Thus, the vertebral stabilization system 10, including an insert 38 contacting a connecting member 22 having a diameter less than 5.5 millimeters (e.g., about 5.0 millimeters or less, about 4.5 millimeters or less, or 4.0 millimeters or less) may have a fatigue strength greater than commercially available vertebral stabilization systems including a spinal rod of a diameter of 5.5 millimeters or greater formed of stainless steel, commercially pure (CP) titanium, alpha-beta titanium alloy (i.e., Ti-6Al-4V or Ti-6Al-7Nb), or cobalt-chromium-molybdenum alloy (Co—Cr—Mo) in direct contact with a threaded fastener (e.g., a set screw). For instance, it has been determined that the connecting member 22 of a diameter of 4.5 millimeters or less secured to the head portion 14 of the vertebral anchor 12 with the securing member 20 has a fatigue strength greater than a spinal rod of a diameter of 5.5 millimeters formed of stainless steel, commercially pure (CP) titanium, Ti-6Al-4V alpha-beta titanium alloy, Ti-6Al-7Nb alpha-beta titanium alloy, or cobalt-chromium-molybdenum alloy (Co—Cr—Mo) secured to a bone screw with a set screw in direct contact with the spinal rod.

The vertebral stabilization system 10 may also include one or more, or a plurality of stabilization members or connecting members 22 extending between vertebral anchors 12 of the vertebral stabilization system 10. As an illustrative example, the vertebral stabilization system 10 shown in FIGS. 1 and 2 includes a first connecting member 22a extending between and secured to the first vertebral anchor 12a and the second vertebral anchor 12b, and a second connecting member 22b extending between and secured to the third vertebral anchor 12c and the fourth vertebral anchor 12d.

As shown in FIGS. 1 and 2, in some embodiments the connecting member 22 may have a uniform cross-sectional dimension (e.g., diameter) along the entire length of the connecting member 22. However, in other embodiments, the connecting member 22 may include one or more regions having a cross-sectional dimension (e.g., diameter) different from the cross-sectional dimension (e.g., diameter) of one or more other regions of the connecting member 22. For instance, in some embodiments the connecting member 22 may include a first region, such as a first end region, configured to be received in the channel 30 of the head portion 14 of the first vertebral anchor 12a which has a first cross-sectional dimension (e.g., diameter), and the connecting member 22 may include a second region, such as a second end region, configured to be received in the channel 30 of the head portion 14 of the second vertebral anchor 12b which has a second cross-sectional dimension (e.g., diameter) which may be the same or different from the first cross-sectional dimension (e.g. diameter). The connecting member 22 may include a third region, such as an intermediate region between the first end region and the second end region, positionable between the head portions 14 of the first and second vertebral anchors 12a/12b which has a third cross-sectional dimension (e.g., diameter) which may be the same or different from the first cross-sectional dimension (e.g. diameter) and/or the second cross-sectional dimension (e.g., diameter). In some instances, the cross-section of the third region, or intermediate region, may be circular or non-circular. For instance, in some cases the third region, or intermediate region, may have a flattened, oval, elliptical, or rectangular cross-section having a cross-sectional dimension in a first direction which is greater than a cross-sectional dimension in a second direction perpendicular to the first direction. Such an embodiment may provide the connecting member 22 with preferential bending in a first plane relative to bending in a second plane perpendicular to the first plane. For instance, if the connecting member 22 were oriented with the larger cross-sectional dimension in a medial-lateral plane and the smaller cross-sectional dimension in an anterior-posterior plane, the connecting member 22 may more readily bend in the anterior-posterior plane than in the medial-lateral plane. Vise versa, if the connecting member 22 were oriented with the smaller cross-sectional dimension in a medial-lateral plane and the larger cross-sectional dimension in an anterior-posterior plane, the connecting member 22 may more readily bend in the medial-lateral plane than in the anterior-posterior plane. In some instances, the first and second end regions of the connecting member 22 may have a diameter of about 5.5 millimeters compatible with commercially available vertebral anchors, while the intermediate region may have a cross-sectional dimension (e.g., diameter) less than 5.5 millimeters, such as between about 4.5 millimeters to about 3.25 millimeters, or about 4.5 millimeters, about 4.0 millimeters, about 3.75 millimeter, or about 3.25 millimeters to increase the flexibility of the connecting member 22. The connecting members 22 will be further discussed later herein.

The vertebral stabilization system 10 may desirably be used in the lumbar region of the spinal column. As used herein, the lumbar region includes the L5-S1 vertebral segment between the L5 lumbar vertebra and the S1 sacrum. However, in some instances the vertebral stabilization system 10 may be used in other regions of the spinal column, such as the cervical, thoracic and thoracolumbar regions. The vertebral stabilization system 10 may be installed multi-laterally on opposite sides of the sagittal plane of the spinal column, with the first and second anchors 12a, 12b and the first connecting member 22a positioned on one lateral side of the sagittal plane and the third and fourth vertebral anchors 12c, 12d and the second connecting member 22b positioned on the other lateral side (i.e., contra-lateral side) of the sagittal plane. However, in other instances the vertebral stabilization system 10, including the first and second vertebral anchors 12a, 12b and the first connecting member 22a, may be installed unilaterally (i.e., on a single side) on the spinal column. Additional vertebral anchors 12 and/or connecting members 22 may be used as desired to support vertebral segments of the spinal column as desired.

The connecting members 22 of the vertebral stabilization system 10 may have stiffness and strength characteristics different from commercially available spinal rods. Commonly used materials for commercially available rigid spinal rods for use in the lumbar region of the spinal column include stainless steel, commercially pure (CP) titanium, alpha-beta titanium alloy (i.e., Ti-6Al-4V or Ti-6Al-7Nb), and cobalt-chromium-molybdenum alloy (Co—Cr—Mo). Due to their relatively high rigidity compared to the natural spine, these systems have been referred to as a “rigid system”.

However, it has been found that the use of a rigid system may lead to adverse effects to the spinal column. For instance, it is believed that the high degree of stiffness of rigid systems may relate to increased stress on adjacent discs and facet joints. Over time, these increased stresses may lead to segment hypermobility, facet hypertrophy, osteophyte formation, and stenosis or so called adjacent level disease.

Recently, less rigid polymeric rod systems have been introduced as an alternative to potentially reduce the stress on adjacent discs and facet joints and the incidences of adjacent level disease. Commonly used materials for these polymer rod systems include polyether ether ketone (PEEK), PEEK composites, or other polymer materials. Due to their relatively high flexibility compared to a rigid system, these systems have been referred to as a “flexible system”. These systems, while more flexible than a rigid system, present their own shortcomings, such as the relatively low ultimate strength of the polymeric materials.

Table 1, below, compares the stiffness (in terms of the elastic modulus) of these commercially available spinal rod materials.

TABLE 1
Elastic Modulus/Stiffness of Commonly Used Rod Materials

| | | | | | | | Short | Continuous |
|---|---|---|---|---|---|---|---|---|
| | | | | | | | Carbon | Carbon |
| | | | | | | | Fibre | Fibre |
| | | | CP | Stainless | | | Reinforced | Reinforced |
| Material | Ti—6Al—4V | Ti—6Al—7Nb | Titanium | steel | Co—Cr—Mo | PEEK | PEEK | PEEK |
| Elastic | 110 | 110 | 105 | 220 | 220 | 4.1 | 17 | 65 |
| Modulus | | | | | | | | |
| (GPa) | | | | | | | | |

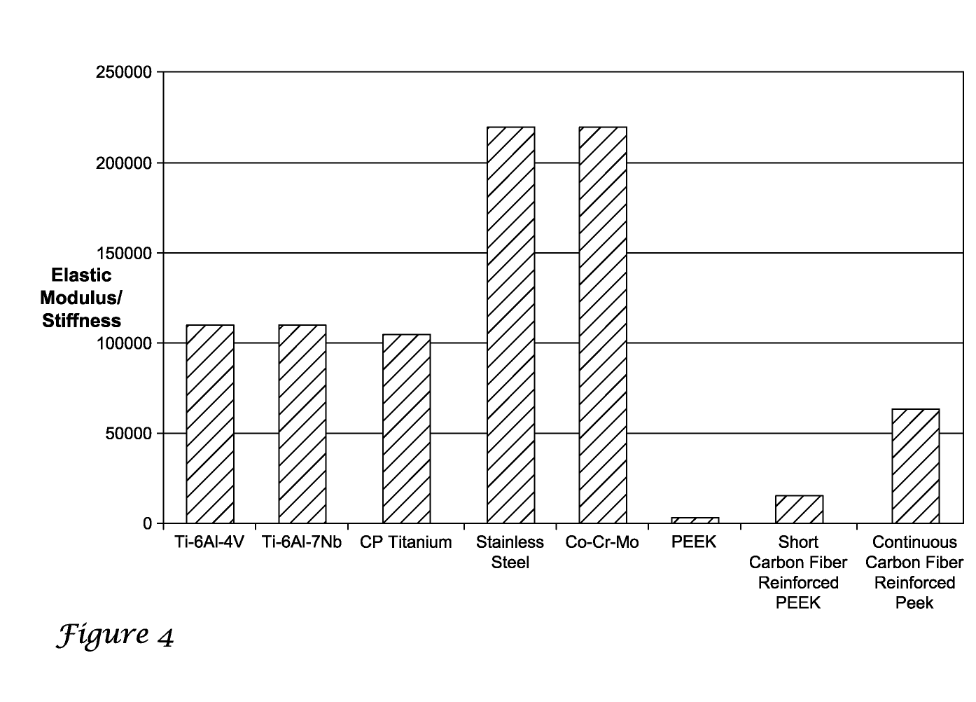

FIG. 4 is a chart comparing the elastic modulus of these commercially available spinal rod materials.

The primary loading mode of spinal rods implanted in spinal fixation systems is caused by axial spine compression loads. Thus, a spinal rod with less structural bending stiffness will deform or bend more than a spinal rod with a greater structural bending stiffness, thus will shift a greater proportion of the spine compression load anteriorly to the vertebral bodies of the spinal segment. Additionally, the loading on the vertebral anchor (e.g., pedicle screw) will be distributed more evenly and reduce the stress at the bone/screw interface. The structural bending stiffness determines a spinal rod's bending flexibility and its load sharing characteristics. Regardless of the material of the spinal rod, a spinal rod having less structural bending stiffness will shift more of the spine compressive load anteriorly and reduce the stress at the interface between the bone and the vertebral anchor.

The structural bending stiffness (flexural strength) of a spinal rod having a circular cross-section can be calculated as:

$$K = EI = E \frac{π {d}^{4}}{64} \qquad ( 1 )$$

where, E is the elastic modulus of the material, I is the moment of the inertia, d is the diameter of the rod. Based on Equation (1), the structural bending stiffness of commercially available rods can be calculated and their values are listed below in Table 2.

TABLE 2
Structural Bending Stiffness of Commonly Used Rods

| | | | CP | Stainless | |
|---|---|---|---|---|---|
| Material | Ti—6Al—4V | Ti—6Al—4V | Titanium | Steel | Co—Cr—Mo |
| Rod | 5.5 | 5.5 | 5.5 | 5.5 | 5.5 |
| diameter | | | | | |
| (mm) | | | | | |
| Elastic | 110 | 110 | 105 | 220 | 220 |
| Modulus | | | | | |
| (GPa) | | | | | |
| Bending | 4,938,478 | 4,938,478 | 4,714,002 | 9,876,956 | 9,876,956 |
| stiffness | | | | | |
| (N-mm²) | | | | | |
| Percentage | 100% | 100% | 95.5% | 200% | 200% |
| of | | | | | |
| Ti—6Al—4V | | | | | |
| 5.5 mm | | | | | |
| rod | | | | | |

| | | | Short | Continuous |
|---|---|---|---|---|
| | | | Carbon | Carbon |
| | | | Fibre | Fibre |
| | | | Reinforced | Reinforced |
| Material | PEEK | PEEK | PEEK | PEEK |
| Rod | 6.35 | 5.5 | 5.5 | 5.5 |
| diameter | | | | |
| (mm) | | | | |
| Elastic | 4.1 | 4.1 | 17 | 65 |
| Modulus | | | | |
| (GPa) | | | | |
| Bending | 327,061 | 184,071 | 763,219 | 2,918,192 |
| stiffness | | | | |
| (N-mm²) | | | | |
| Percentage | 6.6% | 3.7% | 15.5% | 59.1% |
| of | | | | |
| Ti—6Al—4V | | | | |
| 5.5 mm | | | | |
| rod | | | | |

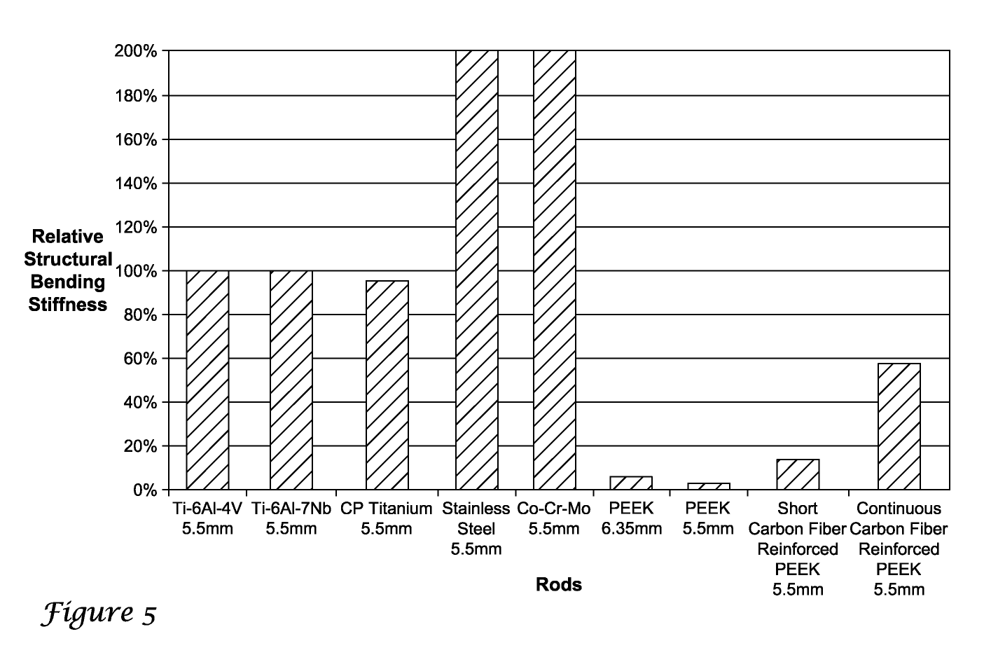

FIG. 5 is a chart illustrating the relative structural bending stiffness of these commercially available spinal rods of various materials as a percentage of structural bending stiffness of a 5.5 millimeter rod formed of Ti-6Al-4V.

As can be seen, the commercially available polymeric rod systems have a considerably reduced structural bending stiffness compared to the conventional rigid rod systems. However, the material strength of the polymeric rods may be deficient in at least some applications. The ultimate strength of the polymeric rod systems is significantly less than the ultimate strength of the conventional rigid rod systems. Table 3, below, compares the ultimate strength of these commercially available spinal rod materials.

TABLE 3
Material Ultimate Strength

| | | | | | | | | Continuous |
|---|---|---|---|---|---|---|---|---|
| | | | | | | | Short Carbon | Carbon |
| | | | | | | | Fibre | Fibre |
| | | | CP | Stainless | | | Reinforced | Reinforced |
| Material | Ti—6Al—4V | Ti—6Al—7Nb | Titanium | steel* | Co—Cr—Mo | PEEK | PEEK | PEEK |
| Ultimate | 896 | 893 | 550 | 965 | 1200 | 110 | 220 | 710 |
| strength | | | | | | | | |
| (MPa) | | | | | | | | |

*Note: The ultimate strength of SS can vary from 90 to 190 ksi (620.5 to 1310 MPa) due to different cold work. The presented data is for SS with 40% cold work.

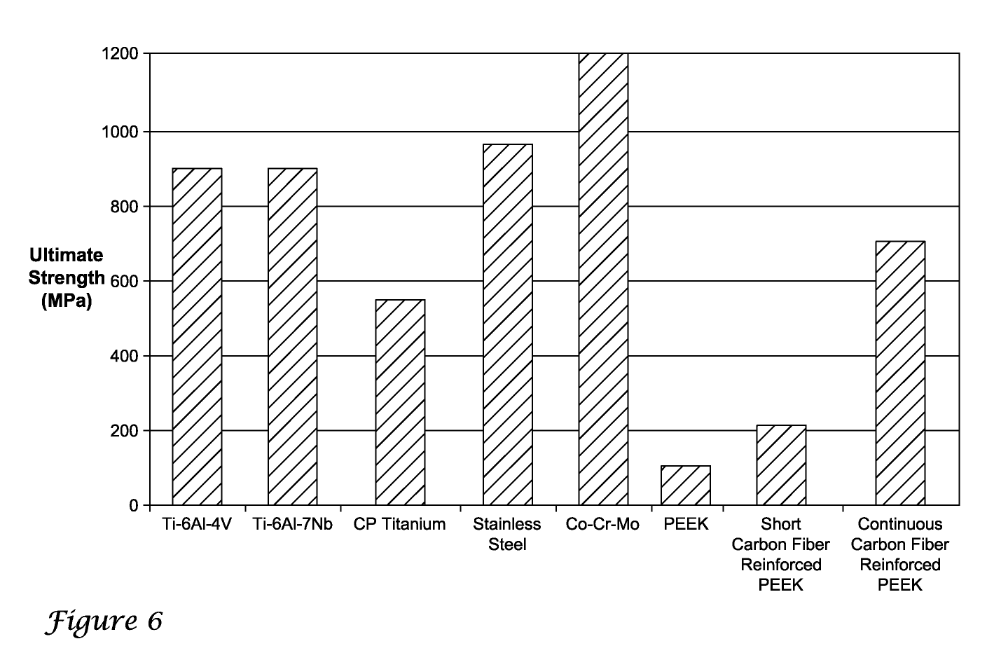

FIG. 6 is a chart comparing the ultimate strength of these commercially available spinal rod materials.

The use of polymeric rods presents additional concerns. For example, carbon debris generated from carbon fiber reinforced PEEK materials may cause biological concerns. Furthermore, unlike conventional metallic rods, polymeric rods can not be bent intra-operatively, preventing the use of polymeric rods in certain applications where rod manipulation (e.g., bending) may be desired or necessary. This may be especially true in multi-level procedures where spinal rods often need to be bent to follow the curvature of the spinal column. For these reasons, currently marketed polymeric rod systems are only indicated for single or two level procedures. In addition, transverse connectors typically can not be used in polymeric rod systems which may prevent the use of transverse connectors in applications in which additional torsion stiffness is needed or desired.

The major loading mode of spinal rods of spinal stabilization systems is bending caused by axial spine compression loads. As such, a spinal rod with less structural bending stiffness will deform more, and thus shift more load anteriorly to the vertebral bodies of the vertebrae. Research data on the load sharing characteristics of a natural spine has determined that the anterior elements carry about 82% of the total spinal compressive load, while the posterior elements carry about 18% of the total spinal compressive load. The anterior elements of the spinal column include the vertebral bodies and vertebral discs of the vertebral segment, while the posterior elements of the spinal column include the facet joints, the spinal cord and foremen of the spinal vertebral segment.

Data has indicated that at spinal segments having spinal stabilization systems utilizing 5.5 millimeter spinal rods formed of Ti-6Al-4V, the posterior elements carry about 30% of the total spinal compressive load, while the anterior elements carry about 70% of the total spinal compressive load. Additional data has indicated that at spinal segments having spinal stabilization systems utilizing 5.5 millimeter spinal rods formed of PEEK, the posterior elements carry about 15% of the total spinal compressive load, while the anterior elements carry about 85% of the total spinal compressive load. Thus, it can be seen that commercially available 5.5 millimeter rods formed of Ti-6Al-4V place too much of the spinal compressive load on the posterior elements, while commercially available 5.5 millimeter rods formed of PEEK shift too much of the spinal compressive loads to the anterior elements of the spinal column.

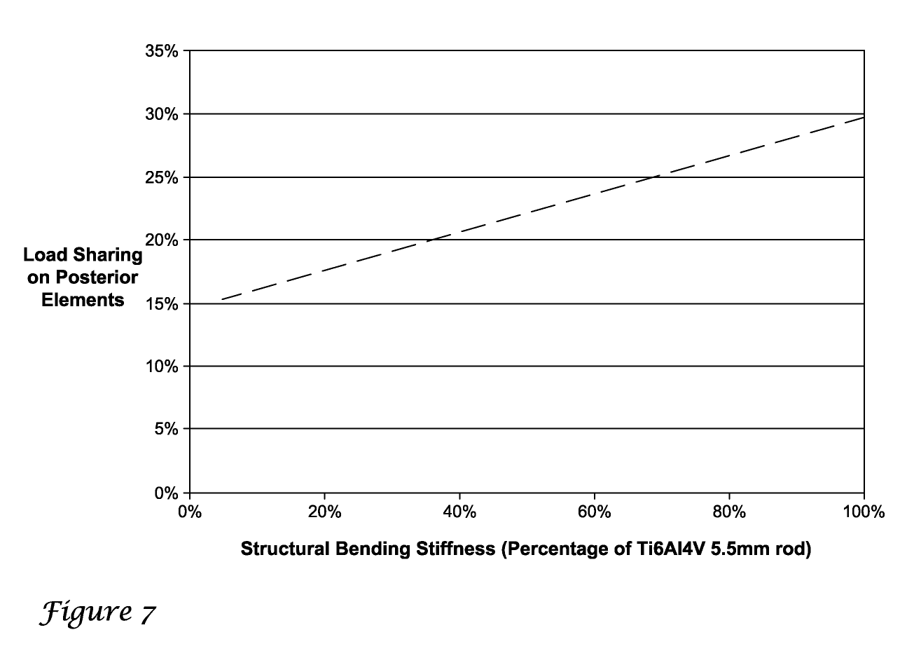

The chart at FIG. 7 illustrates the percent of load sharing of an axial spine compression load of 5.5 millimeter spinal rods formed of PEEK and Ti-6Al-4V relative to the structural bending stiffness of the spinal rod as a percentage of the structural bending stiffness of a 5.5 millimeter spinal rod formed of Ti-6Al-4V. As can be seen from FIG. 7, the posterior elements carry about 30% of the total spinal compressive load at spinal segments having spinal stabilization systems utilizing 5.5 millimeter spinal rods formed of Ti-6Al-4V, whereas the posterior elements carry about 15% of the total spinal compressive load at spinal segments having spinal stabilization systems utilizing 5.5 millimeter spinal rods formed of PEEK.

Unlike the conventional spinal rod constructs described herein, the connecting member 22, described herein, may have a structural bending stiffness (flexural strength) less than commercially available metallic rods, but greater than the structural bending stiffness (flexural strength) of commercially available polymeric rods. For example, the structural bending stiffness of the connecting member 22 may be between about 10% to about 40%, about 15% to about 30%, about 20% to about 25%, or about 25% of the structural bending stiffniess of a commercially available 5.5 millimeter Ti-6Al-4V spinal rod. In some instances, the structural bending stiffness of the connecting member 22 may be about 500,000 N-mm² to about 2,000,000 N-mm², about 500,000 N-mm² to about 1,250,000 N-mm², about 1,000,000 N-mm² to about 2,000,000 N-mm², about 500,000 N-mm², about 550,000, about 1,000,000 N-mm², about 1,250,000 N-mm², or about 2,000,000 N-mm².

Thus, the use of a connecting member 22 having these characteristics in a spinal stabilization system at a spinal segment may more closely approximate the load sharing characteristics of a natural spine, such that the posterior elements carry about 18% of the total spinal compressive load, while the anterior elements carry about 82% of the total spinal compressive load. In some instances, between about 17% to about 19%, or about 18% of the total compressive load of a spinal segment having a spinal stabilization system 10 utilizing connecting members 22, described herein, may be transferred through the posterior elements, while between about 81% to about 83%, or about 82% of the total compressive load may be transferred through the anterior elements of the spinal segment.

The distribution of the spine compressive load on the anterior and posterior elements of a spinal segment may be controlled by controlling the diameter of the connecting member 22 and/or the material of the connecting member 22. Accordingly, the connecting member 22 may have a diameter less than 5.5 millimeters, for instance a diameter of about 5.0 millimeters or less or about 4.5 millimeters or less. For example, the connecting member 22 may have a diameter ranging from about 3.25 millimeters to about 4.5 millimeters. The connecting member 22 may be formed of a material, such as a metallic alloy, which has an ultimate strength greater than 1 GPa and an elastic modulus of less than or equal to 110 GPa. In some instances, the connecting member 22 may be formed of a material having an ultimate strength greater than 1 GPa and an elastic modulus of less than or equal to 100 GPa. One such material is a high strength beta titanium alloy, namely Ti-15Mo-5Zr beta titanium alloy having an elastic modulus of about 99 GPa and an ultimate strength of about 1.5 GPa. As can be seen from Table 4, below, high strength Ti-15Mo-5Zr beta titanium alloy has an elastic modulus less than that of conventional Ti-6Al-4V alpha-beta titanium alloy, but has a higher ultimate strength than Ti-6Al-4V.

TABLE 4
Material Comparison

| | | | | | | | Short Carbon | Continuous | |
|---|---|---|---|---|---|---|---|---|---|
| | | | | | | | Fibre | Carbon Fibre | |
| | | | | Stainless | | | Reinforced | Reinforced | High strength |
| Material | Ti—6Al—4V | Ti—6Al—7Nb | CP Titanium | steel | Co—Cr—Mo | PEEK | PEEK | PEEK | Ti—15Mo—5Zr |
| Elastic | 110 | 100 | 105 | 220 | 220 | 4.1 | 17 | 65 | 99 |
| Modulus | | | | | | | | | |
| (GPa) | | | | | | | | | |
| Ultimate | 896 | 893 | 550 | 965 | 1200 | 110 | 220 | 710 | 1468 |
| Strength (MPa) | | | | | | | | | |

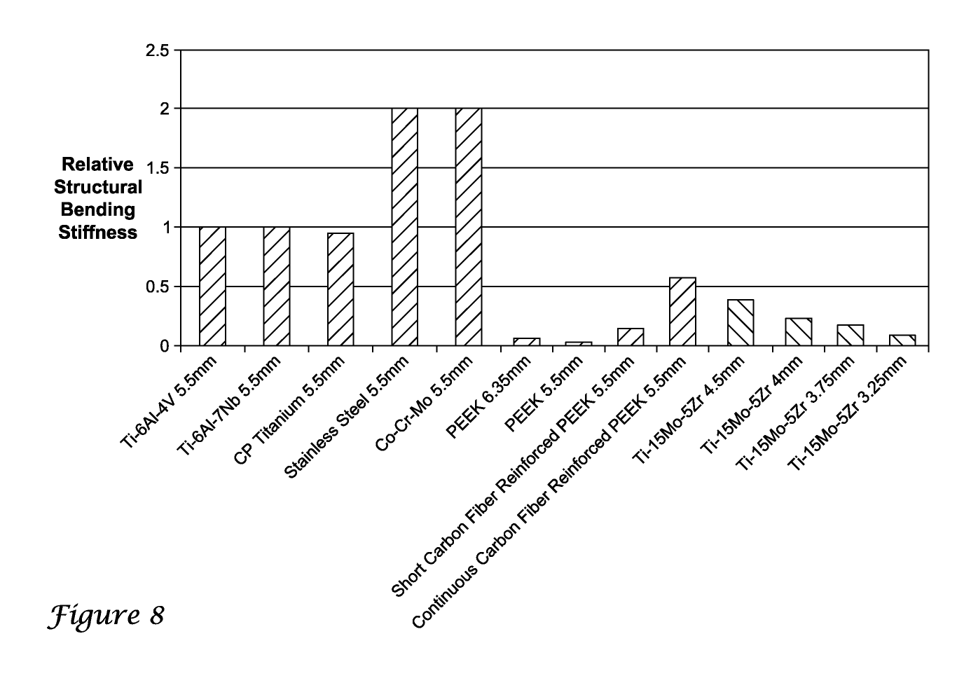

Table 5, below, and FIG. 8 illustrate the structural bending stiffness of connecting members 22 having diameters of 3.25 millimeters, 3.75 millimeters, 4 millimeters, and 4.5 millimeters formed of high strength beta titanium alloy Ti-15Mo-5Zr material.

TABLE 5
Structural Bending Stiffness of Various
Sized Rods formed of Ti—15Mo—5Zr

| | Rod diameter (mm) | | | |
|---|---|---|---|---|
| | 3.25 | 3.75 | 4.0 | 4.5 |
| Elastic | 99 GPa | 99 GPa | 99 GPa | 99 GPa |
| Modulus | | | | |
| Bending | 541,899 | 960,528 | 1,243,440 | 1,991,750 |
| stiffness | | | | |
| (N-mm²) | | | | |
| Percentage of | 10.9% | 19.4% | 25.2% | 40.3% |
| Ti—6Al—4V | | | | |
| 5.5 mm rod | | | | |

As can be seen from Table 5, the connecting member 22 formed of high strength beta titanium alloy Ti-15Mo-5Zr material and having a diameter of 4 millimeters has a structural bending stiffness of about 1,250,000 N-mm² or about 25% of the structural bending stiffness of a 5.5 millimeter spinal rod formed of Ti-6Al-4V. Thus, the use of a 4.0 millimeter diameter connecting member 22 formed of Ti-15Mo-5Zr in a spinal stabilization system at a spinal segment may more closely approximate the load sharing characteristics of a natural spine, such that the posterior elements carry about 18% of the total spinal compressive load, while the anterior elements carry about 82% of the total spinal compressive load. Furthermore, the connecting member 22 formed of high strength beta titanium alloy Ti-15Mo-5Zr material and having a diameter of 3.25 millimeters has a structural bending stiffness of about 500,000 N-mm² or about 10% of the structural bending stiffness of a 5.5 millimeter spinal rod formed of Ti-6Al-4V, the connecting member 22 formed of high strength beta titanium alloy Ti-15Mo-5Zr material and having a diameter of 3.75 millimeters has a structural bending stiffness of about 1,000,000 N-mm² or about 20% of the structural bending stiffness of a 5.5 millimeter spinal rod formed of Ti-6Al-4V, and the connecting member 22 formed of high strength beta titanium alloy Ti-15Mo-5Zr material and having a diameter of 4.5 millimeters has a structural bending stiffness of about 2,000,000 N-mm² or about 40% of the structural bending stiffness of a 5.5 millimeter spinal rod formed of Ti-6Al-4V.

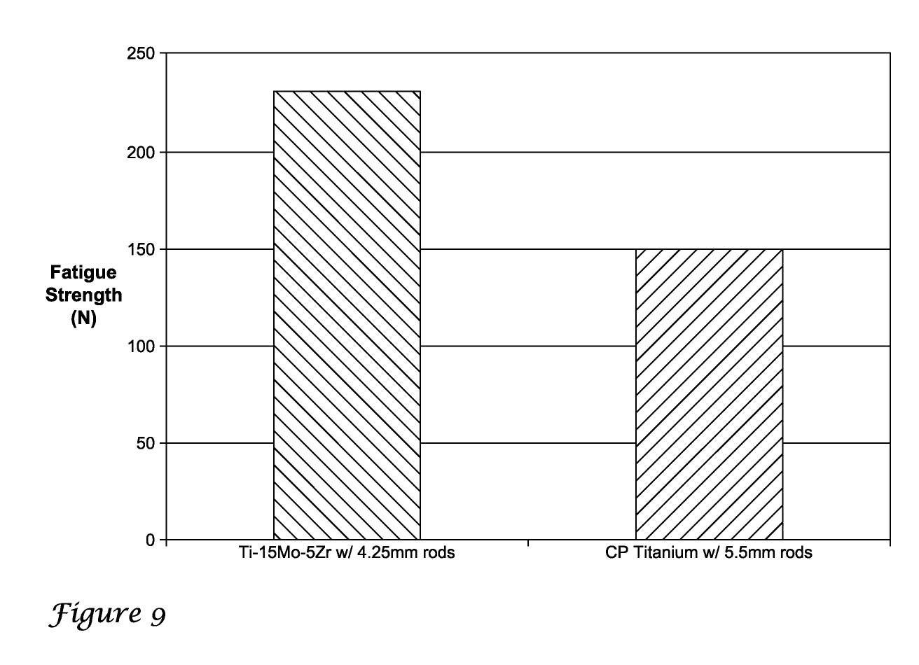

Experimental testing following ASTM F1717-04 has been conducted for a 4.25 millimeter diameter rod formed of Ti-15Mo-5Zr. The test results indicated that the test rod withstood a 230 N load for 5,000,000 cycles. Earlier test results showed a commercially available 5.5 millimeter rod formed of CP titanium had a run-out load of 150 N. Thus, it was determined that the fatigue strength of the 4.25 millimeter diameter rod formed of Ti-15Mo-5Zr (230 N) was about a 50% greater than the fatigue strength of the 5.5 millimeter rod formed of CP titanium (150 N). A comparison of the fatigue strength of the 4.25 millimeter diameter rod formed of Ti-15Mo-5Zr to the fatigue strength of the 5.5 millimeter rod formed of CP titanium is shown at FIG. 9.

In some instances it may be necessary or desirable to install a first connecting member 22 of a first structural bending stiffness on a first, lateral side of the spinal column while a second connecting member 22 of a second structural bending stiffness different from the first structural bending stiffness is installed on a second, contra-lateral side of the spinal column. For instance, the first connecting member 22 may have a first diameter and the second connecting member 22 may have a second diameter different from the first diameter, while the material of the first and second connecting members 22 is the same or different. In other instances, the first connecting member 22 may be formed of a first material having a first elastic modulus and the second connecting member 22 may be formed of a second material having a second elastic modulus different from the first elastic modulus, while the diameter of the first and second connecting members 22 is the same or different. Such a selection may provide a surgeon a choice to match the desired bending stiffness and/or load distribution for a specific patient.

The connecting members 22, as described herein, may be advantageous over a commercially available 5.5 millimeter diameter metallic rod system, as the connecting members 22 have a lower structural bending stiffness with an equal or greater fatigue strength than a commercially available 5.5 millimeter diameter metallic rod. Furthermore, the connecting members 22, as described herein, may be advantageous over a commercially available 5.5 millimeter diameter polymeric rod system, as the connecting members 22 have a higher ultimate strength while maintaining a comparable structural bending stiffness. Thus, a connecting member 22, as described herein, which has a diameter of less than 5.5 millimeters may be used in the lumbar region of a patient, where contemporary understanding indicates that commercially available 5.5 millimeter rods are required.

Additional benefits of the connecting member 22 having these characteristics include the ability of the connecting member 22 to be bent intra-operatively allowing the use of the connecting member 22 in applications where rod manipulation (e.g., bending) may be desired or necessary, such as lordosed regions of the lumbar region of the spinal column. Furthermore, transverse connectors, such as the transverse connector 60 shown in FIG. 1, can be used with the connecting member 22 having these characteristics in applications in which additional torsion stiffness is needed or desired, or otherwise where the use of transverse connectors may be desired. The connecting members 22 are also radio-opaque.

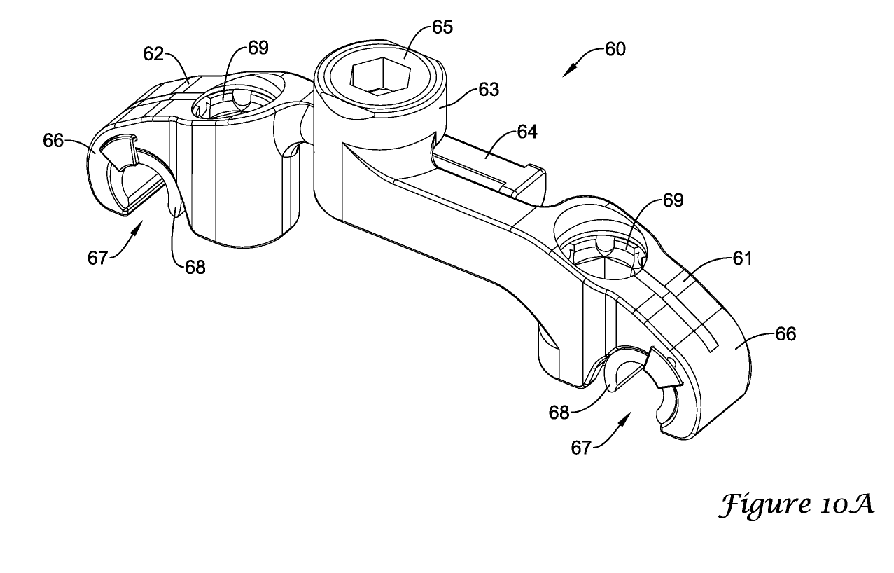

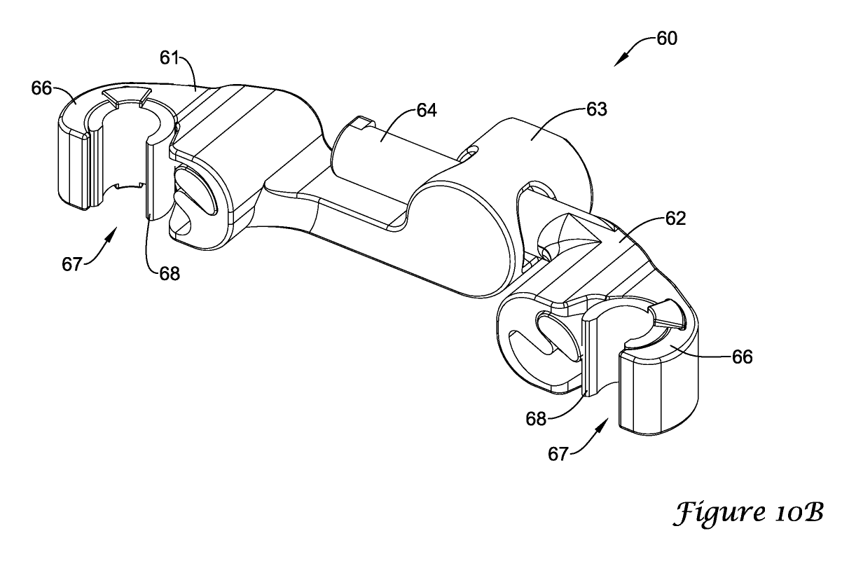

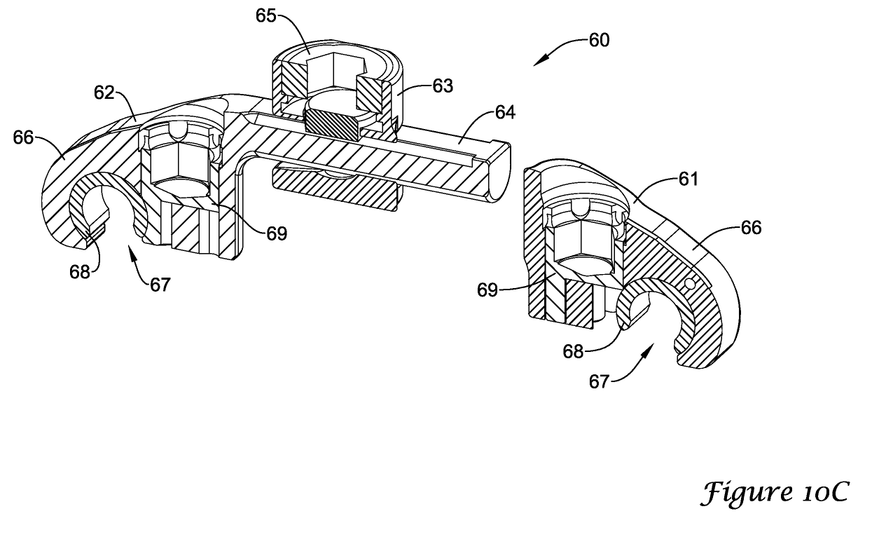

The transverse connector 60 is further illustrated at FIGS. 10A, 10B and 10C. The transverse connector 60, in many respects, is similar to the transverse connector disclosed in U.S. Pat. No. 7,485,132, incorporated herein by reference. The transverse connector 60 may include a first section 61 and a second section 62 selectively coupled to the first section 61. For instance, the first section 61 may be coupled to the second section 62 at any of a plurality of longitudinal and/or angular positions. For example, the first section 61 may include a housing 63 configured to receive a rod 64 of the second section 62 therein. The rod 64 may be secured in the housing 63 with a fastener 65 at any of a plurality of longitudinal and/or angular positions. Each of the first and second sections 61, 62 may include a rod coupling region 66 configured to surround a portion of an connecting member 22.

An insert 68 may be positioned in the opening of the rod coupling region 66 for spacing the rod coupling region 66 of the transverse connector 60 from direct contact with the connecting member 22. The insert 68, which may be a C-shaped member or similarly shaped, may include a channel 67 extending therethrough into which a connecting member 22 may be positioned. The transverse connecter 60 may also include cam members 69 which may be rotated to secure the transverse connector 60 to the connecting member 22.

The insert 68 may be formed of a material having a lower modulus of elasticity than the material forming the rod coupling regions 66 and the cam members 69 of the transverse connector 60. For instance, the rod coupling regions 66 and/or the cam members 69 may be formed of a metallic material, such as stainless steel, CP titanium, titanium alloy, cobalt-chromium-molybdenum alloy (Co—Cr—Mo), or other biocompatible metallic material. The insert 68 may be formed of a polymeric material, such as polyether ether ketone (PEEK), carbon fiber reinforced PEEK, ultra high molecular weight polyethylene (UHMWPE), or poly(methyl methacrylate) (PMMA).

In order to secure the connecting member 22 to the transverse connector 60, the cam members 69, or other securing members, may be rotated with a driving tool, which exerts a force onto the connecting members 22 positioned in the channel 67 via the inserts 68. The presence of the inserts 68 between the cam members 69 and the connecting members 22 may provide a stress gradient between the cam member 69 and the connecting members 22, distributing the forces exerted onto the connecting members 22 through tightening the cam members 69 over a larger portion of the exterior surface of the connecting members 22. Therefore, the inclusion of the inserts 68 may eliminate any notching of the connecting members 22, thereby increasing the fatigue strength of the connecting members 22 in the vertebral stabilization system 10 such that a connecting member 22 of a smaller diameter than conventional spinal rods (e.g., smaller than 5.5 millimeters) may be used in the vertebral stabilization system 10 while not compromising the fatigue strength of the connecting member 22 and the vertebral stabilization system 10.

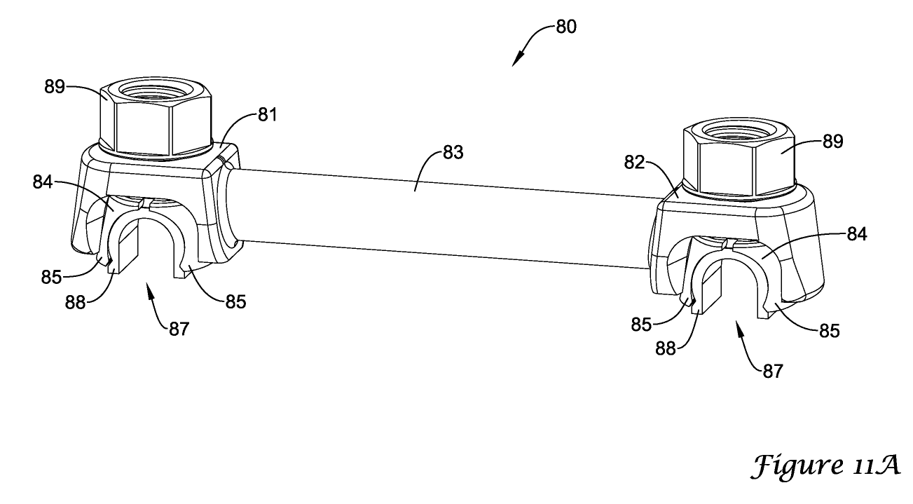

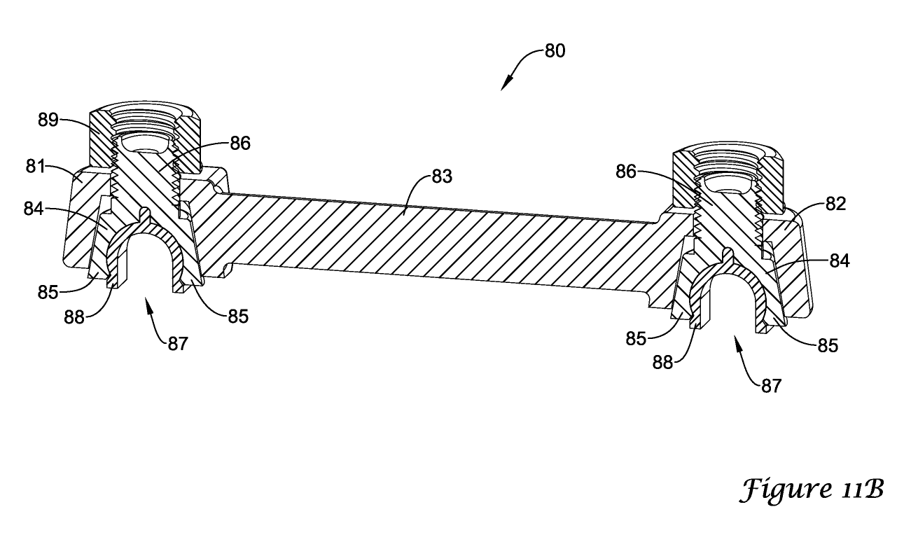

An alternative embodiment of a transverse connector for use in the vertebral stabilization system 10 is illustrated in FIGS. 11A and 11B. The transverse connector 80, in many respects, is similar to the transverse connector disclosed in U.S. Pat. No. 6,328,740, incorporated herein by reference. The transverse connector 80 may include first and second housings 81, 82 coupled together with a linking portion 83. Each of the first and second housings 81, 82 is configured to be coupled to a connecting member 22 of the vertebral stabilization system 10. A rod coupling member 84 may be positioned in an opening of each of the first and second housings 81, 82. The rod coupling member 84 may include first and second legs 85 which may be deflectable toward one another upon the application of force.

An insert 88 may be positioned in the opening of the rod coupling member 84 between the first and second legs 85 for spacing the rod coupling member 84 of the transverse connector 80 from direct contact with the connecting member 22. The insert 88, which may be a C-shaped member or similarly shaped, may include a channel 87 extending therethrough into which a connecting member 22 may be positioned. The transverse connecter 80 may also include threaded nuts 89 threadably engaging threaded shafts 86 of the rod coupling members 84 which may be rotated to draw the coupling members 84 into the first and second housings 81, 82 to secure the transverse connector 80 to the connecting member 22.

The insert 88 may be formed of a material having a lower modulus of elasticity than the material forming the rod coupling members 84 or other components of the transverse connector 80. For instance, the rod coupling members 84 and/or other components of the transverse connector 80 may be formed of a metallic material, such as stainless steel, CP titanium, titanium alloy, cobalt-chromium-molybdenum alloy (Co—Cr—Mo), or other biocompatible metallic material. The insert 88 may be formed of a polymeric material, such as polyether ether ketone (PEEK), carbon fiber reinforced PEEK, ultra high molecular weight polyethylene (UHMWPE), or poly(methyl methacrylate) (PMMA).

In order to secure the connecting member 22 to the transverse connector 80, the threaded nuts 89 may be rotated with a driving tool, drawing the rod coupling members 84 into the housings 81, 82. As the rod coupling members 84 are drawn into the housings 81, 82, the legs 85 of the rod coupling members 84 are deflected toward one another due to the engagement between the rod coupling members 84 and the housings 81, 82. As the legs 85 are deflected, a clamping force is exerted on the connecting member 22 via the insert 88. The presence of the inserts 88 between the rod coupling members 84 and the connecting members 22 may provide a stress gradient between the rod coupling members 84 and the connecting members 22, distributing the forces exerted onto the connecting members 22 through tightening the threaded nuts 89 over a larger portion of the exterior surface of the connecting members 22. Therefore, the inclusion of the inserts 88 may eliminate any notching of the connecting members 22, thereby increasing the fatigue strength of the connecting members 22 in the vertebral stabilization system 10 such that a connecting member 22 of a smaller diameter than conventional spinal rods (e.g., smaller than 5.5 millimeters) may be used in the vertebral stabilization system 10 while not compromising the fatigue strength of the connecting member 22 and the vertebral stabilization system 10.

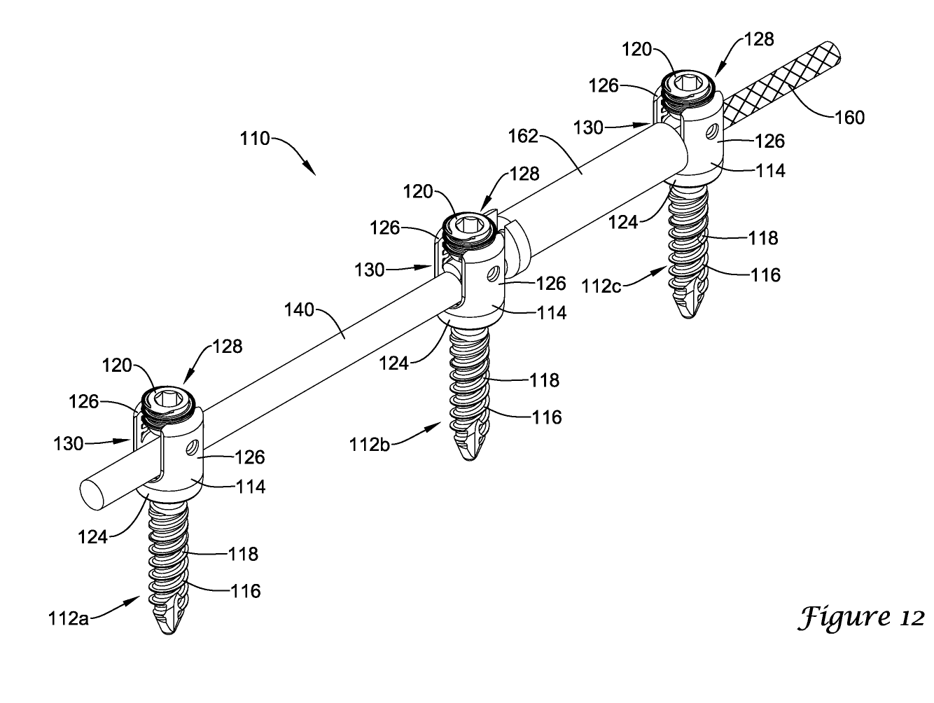

Another illustrative vertebral stabilization system 110 is illustrated at FIG. 12. The vertebral stabilization system 110 may include one or more or a plurality of vertebral anchors or fasteners 112. Although the vertebral anchors 112 are depicted as threaded vertebral fasteners (e.g., pedicle screws, bone screws), in some embodiments the vertebral anchors 112 may be vertebral hooks (e.g., laminar hooks) or other types of fastening members for attachment to a bony structure such as a vertebra of the spinal column. Each of the vertebral anchors 112 may be configured to be secured to a vertebra of a spinal column. For instance, the first vertebral anchor 112a may be secured to a first vertebra, the second vertebral anchor 112b may be secured to a second vertebra, and the third vertebral anchor 112c may be secured to a third vertebra.

The vertebral anchor 112 may include a head portion 114 and a bone engagement portion 116 extending from the head portion 114. In some embodiments, the bone engagement portion 116 may be a shaft portion 118 of the vertebral anchor 112 extending from the head portion 114 along a longitudinal axis of the vertebral anchor 112. In some embodiments, the vertebral anchor 112 may be a monoaxial screw in which the head portion 114 is stationary relative to the shaft portion 118, and in other embodiments the vertebral anchor 112 may be a polyaxial screw in which the head portion 114 is actuatable (e.g., pivotable) relative to the shaft portion 118. In some embodiments, the shaft portion 118 may be configured to be installed into a bony region of a vertebra of the spinal column. For example, the shaft portion 118 may be installed into a pedicle of a vertebra, or other region of a vertebra. In some embodiments, the shaft portion 118 may be a threaded region having helical threads configured to be screwed into a pedicle of a vertebra, or other bony region of a vertebra.

The head portion 114 may include a base portion 124, from which the shaft portion 118 extends from, and first and second legs 126 extending from the base portion 124 on opposing sides of the head portion 114. The first and second legs 126 may define an opening 128, which may be a threaded opening in some instances, extending into the head portion 114 from an upper extent of the head portion 114 opposite the base portion 124. In embodiments in which the opening 128 is threaded, each of the first and second legs 126 may include a threaded portion for threadedly engaging a threaded portion of a securing member 120. In other embodiments, the first and second legs 126 may include other engagement features for engaging with a securing member positioned in the opening 128 between the first and second legs 126. The head portion 114 may additionally include a channel 130, such as a U-shaped channel, defined between the first and second legs 126. The channel 130 may extend through the head portion 114 from a first side of the head portion 114 to a second side of the head portion 114. The opening 128 may intersect the channel 130.

The vertebral anchor 112 may include a securing element, such as a threaded fastener 120 (e.g., a set screw, cap) configured to engage the head portion 114 to secure one or more elongate members to the vertebral anchor 112. For example, the threaded fastener 120 may include threads which mate with threads formed in the head portion 114. In other embodiments, other securing members, having engagement features, may be used to secure one or more elongate members, such as an elongate rod or flexible member, in the head portion 114 of the vertebral anchor 112.

The vertebral stabilization system 110 may also include one or more, or a plurality of elongate connecting members extending between vertebral anchors 112 of the vertebral stabilization system 110. As an illustrative example, the vertebral stabilization system 110 shown in FIG. 12 includes a first elongate member, shown as an elongate rod 140, extending between and secured to the first vertebral anchor 112a and the second vertebral anchor 112b, and a second elongate member, shown as a flexible member 160 (e.g., a flexible cord), extending between and secured to the second vertebral anchor 112b and the third vertebral anchor 112c.

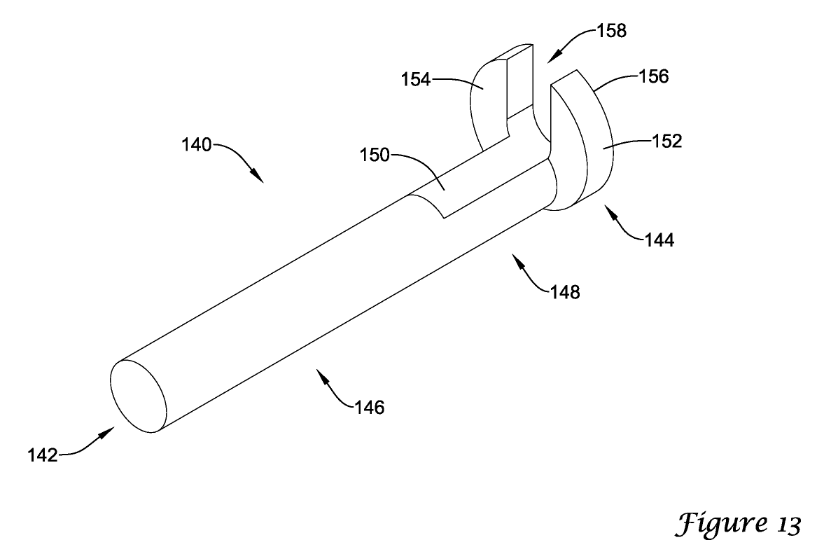

FIG. 13 is a perspective view of the elongate rod 140 of the vertebral stabilization system 110. The elongate rod 140 may have a first end 142, a second end 144, and a length between the first end 142 and the second end 144 sufficient to span the distance between first vertebral anchor 112a and the second vertebral anchor 112b. The elongate rod 140 may be formed of any desired material, including those materials listed above such as stainless steel, commercially pure (CP) titanium, alpha-beta titanium alloy (e.g., Ti-6Al-4V), beta titanium alloy (e.g., Ti-15Mo-5Zr), other metals or metal alloys, polyether ether ketone (PEEK), PEEK composites, or other polymer materials.

The elongate rod 140 may include a first region 146 and a second region 148. The first region 146 may have a circular cross-section having a desired diameter, such as a diameter of about 5.5 millimeters, about 5.0 millimeters, about 4.5 millimeters, about 4.25 millimeters, about 4.0 millimeters, about 3.75 millimeters, or about 3.5 millimeters, in some instances. It is contemplated that the first region 146 may also have a non-circular cross-section in some instances.

In some instances, the second region 148 of the elongate rod 140 may be of a reduced diameter relative to the first region 146. For example, in some instances, the first region 146 may have a diameter of about 5.5 millimeters or more, while the second region 148 may have a diameter of less than 5.5 millimeters, such as about 5.0 millimeters, about 4.5 millimeters, about 4.25 millimeters, about 4.0 millimeters, about 3.75 millimeters, or about 3.5 millimeters. A transition region, such as a tapered region, or a step-wise transition may be located between the first region 146 and the second region 148.

The second region 148 may include at least a portion having an exterior engagement surface 150 against which the flexible member 160 may be positioned adjacent to. In some instances the exterior engagement surface 150, which is a portion of the exterior surface of the elongate rod 140, may be a planar surface. In other instances, the exterior engagement surface 150 may be a slightly convexly curved surface having a radius of curvature different from the radius of curvature of the remainder of the outer surface of the second region 148 of the elongate rod 140. For instance, the radius of curvature of the exterior engagement surface 150 may be greater than the radius of curvature of the outer surface around the circumference of the remainder of the second region 148. Thus, the center of curvature of the exterior engagement surface 150 may be offset from the central longitudinal axis of the elongate rod 140. In other embodiments, as shown in FIG. 13, the exterior engagement surface 150 may be a concave surface on the exterior of the elongate rod 140 forming an open channel along at least a portion of the second region 148 of the elongate rod 140 for placement of a portion of the flexible member 160 there along. Thus, the second region 148 may have a non-circular cross-section throughout at least a portion of the second region 148.

The elongate rod 140 may also include a flange 152 at the second end 144 of the elongate rod 140. The flange 152 may include a first side surface 154 and a second side surface 156 opposite the first side surface 154. In some instances, the flange 152 may be generally circular with a center point coaxial with the central longitudinal axis of the elongate rod 140, while in other instances, the center point of the flange 152 may be off-set from and non-coaxial with the central longitudinal axis of the elongate rod 140. The flange 152 may include an opening, shown in the form of a notch 158, extending toward the center of the flange 152 from the periphery of the flange 152. Thus, the notch 158 may be open to the periphery of the flange 152. The notch 158 may accommodate a portion of the flexible member 160 extending from one side of the flange 152 to the other side of the flange 152 when both the flexible member 160 and the elongate rod 140 are received and secured in the head portion 114 of the second vertebral anchor 112b. Thus, the notch 158 may allow the flexible member 160, extending along side of the elongate rod 140, to remain closer to the central longitudinal axis of the elongate rod 140 as the flexible member 160 extends past the flange 152 toward the third vertebral anchor 112c.

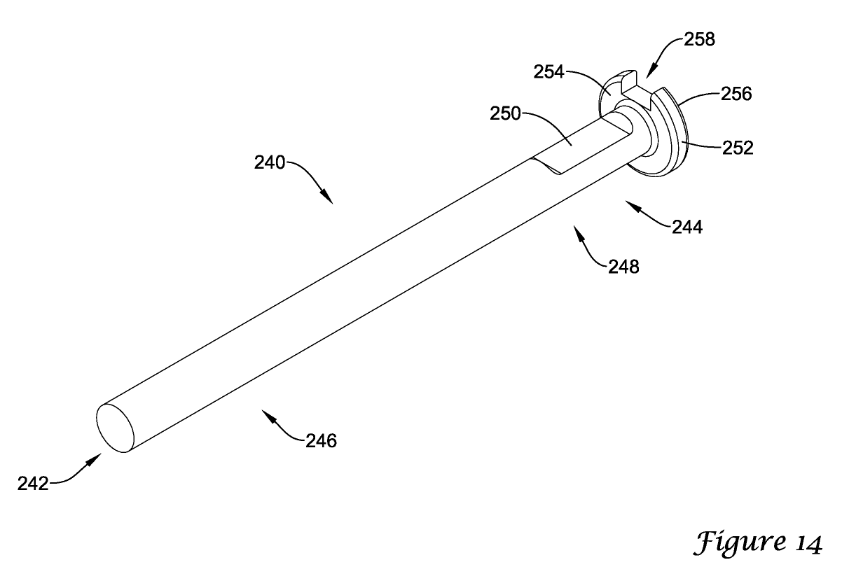

FIG. 14 is a perspective view of an alternate embodiment of an elongate rod 240, similar to the elongate rod 140, which may be used in the vertebral stabilization system 110. For instance, the elongate rod 240 may include a first end 242, a second end 244 and a length between the first end 242 and the second end 244. Additionally, the elongate rod 240 may include a first region 246 and second region 248 which may include at least a portion having an exterior engagement surface 250 against which the flexible member 160 may be positioned adjacent to. In some instances the exterior engagement surface 250, which may be a flattened exterior surface, may be a planar surface. In other instances, the exterior engagement surface 250 may be a slightly curved surface having a radius of curvature different from the radius of curvature of the remainder of the outer surface of the second region 248 of the elongate rod 240. For instance, the radius of curvature of the exterior engagement surface 250 may be greater than the radius of curvature of the outer surface around the circumference of the remainder of the second region 248. Thus, the center of curvature of the exterior engagement surface 250 may be offset from the central longitudinal axis of the elongate rod 240. Thus, the second region 248 may have a non-circular cross-section throughout at least a portion of the second region 248.

In some instances, the second region 248 of the elongate rod 240 may be of a reduced diameter relative to the first region 246. For example, in some instances, the first region 246 may have a diameter of about 5.5 millimeters or more, while the second region 248 may have a diameter of less than 5.5 millimeters, such as about 5.0 millimeters, about 4.5 millimeters, about 4.25 millimeters, about 4.0 millimeters, about 3.75 millimeters, or about 3.5 millimeters. A transition region, such as a tapered region, or a step-wise transition may be located between the first region 246 and the second region 248.

The elongate rod 240, similar to the elongate rod 140, may also include a flange 252 at the second end 244 of the elongate rod 240. The flange 252 may include a first side surface 254 and a second side surface 256 opposite the first side surface 254. In some instances, the flange 252 may be generally circular with a center point coaxial with the central longitudinal axis of the elongate rod 240, while in other instances, the center point of the flange 252 may be off-set from and non-coaxial with the central longitudinal axis of the elongate rod 240. The flange 252 may include an opening, shown in the form of a notch 258, extending toward the center of the flange 252 from the periphery of the flange 252. Thus, the notch 258 may be open to the periphery of the flange 252. The notch 258 may accommodate a portion of the flexible member 160 extending from one side of the flange 252 to the other side of the flange 252 when both the flexible member 160 and the elongate rod 240 are received and secured in the head portion 114 of the second vertebral anchor 112b. Thus, the notch 258 may allow the flexible member 160, extending along side of the elongate rod 240, to remain closer to the central longitudinal axis of the elongate rod 240 as the flexible member 160 extends past the flange 252 toward the third vertebral anchor 112c.

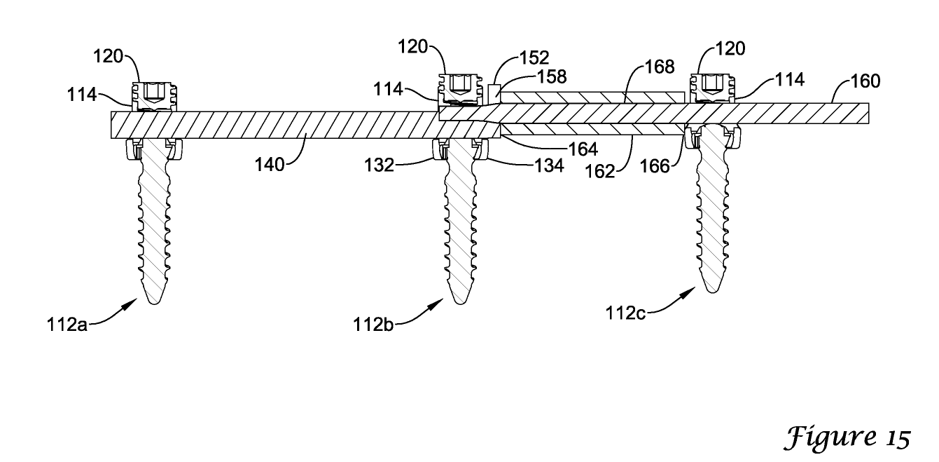

The flexible member 160, which in some instances may be a flexible cord, may be positioned adjacent to the elongate rod 140 in a side-by-side fashion in the head portion 114 of the second vertebral anchor 112b, with a portion of the flexible member 160 overlapping a portion of the elongate rod 140 such that an exterior surface of the flexible member 160 is positioned adjacent to and in contact with an exterior surface of the elongate rod 140. For example, a portion of the flexible member 160 may extend along and contact the exterior engagement surface 150 of the elongate rod 140. With such a configuration, the central longitudinal axis of the elongate rod 140 may be offset from and non-coaxial with the central longitudinal axis of the flexible member 160. FIG. 15 is a longitudinal cross-sectional view of the vertebral stabilization system 110 shown in FIG. 12 which further illustrates the overlapping positioning of the elongate rod 140 and the flexible member 160 in the head portion 114 of the second vertebral anchor 112b.

The elongate rod 140 may be positioned in the channel 130 of the head portion 114 of the second vertebral anchor 112b such that the flange 152 is positioned facing the second side 134 of the head portion 114 with the elongate rod 140 extending from the head portion 114 of the second vertebral anchor 112b to the head portion 114 of the first vertebral anchor 112a. Thus, at least the portion of the second region 148 of the elongate rod 140 including the exterior engagement surface 150 may be positioned within the channel 130 of the head portion 114 of the second vertebral anchor 112b. Thus, a portion of the flexible member 160 overlapping and positioned adjacent the exterior engagement surface 150 of the elongate rod 140 may also be positioned within the channel 130 of the head portion 114 of the second vertebral anchor 112b. The flexible member 160 may extend from the head portion 114 through the notch 158 of the flange 152 to the third vertebral anchor 112c.

A spacer 162 may be disposed on the flexible member 160 and be positioned between the flange 152 and the head portion 114 of the third vertebral anchor 112c. For instance, the spacer 162 may include a first end 164, a second end 166 and a lumen 168 extending through the spacer 162 from the first end 164 to the second end 166. The flexible member 160 may extend through the lumen 168 of the spacer 162. When positioned between the flange 152 and the head portion 114 of the third vertebral anchor 112c, the first end 164 of the spacer 162 may abut or otherwise contact the second side surface 156 of the flange 152 and the second end 166 of the spacer 162 may abut or otherwise contact a side surface of the head portion 114 of the third vertebral anchor 112c.

The threaded fastener 120, or other securing element, may be engaged (e.g., rotatably or threadably engaged) to the head portion 114 to exert a clamping force on the flexible member 160 and the elongate rod 140 to secure the flexible member 160 and the elongate rod 140 in the channel 130 of the head portion 114 of the second vertebral anchor 112b.

As shown in FIG. 15, in some instances the flexible member 160 may be positioned above the elongate rod 140 such that the elongate rod 140 rests against the base portion 124 and the flexible member 160 is positioned between the elongate rod 140 and the threaded fastener 120. Thus, in such instances the threaded fastener 120 may exert a force against the flexible member 160, which in turn exerts a force against the elongate rod 140 to secure the flexible member 160 and the elongate rod 140 in the channel 130 of the head portion 114 of the second vertebral anchor 112b. Positioning the flexible member 160 between the threaded fastener 120 and the elongate rod 140 may protect the elongate rod 140 from a notching effect (e.g., galling/fretting) attributed to direct contact between the threaded fastener 120 and the elongate rod 140 which may in turn increase the fatigue strength of the elongate rod 140. In other instances, the elongate rod 140 may be positioned above the flexible member 160 such that the flexible member 160 rests against the base portion 124 and the elongate rod 140 is positioned between the flexible member 160 and the threaded fastener 120. Thus, in such instances the threaded fastener 120 may exert a force against the elongated rod 140, which in turn exerts a force against the flexible member 160 to secure the flexible member 160 and the elongate rod 140 in the channel 130 of the head portion 114 of the second vertebral anchor 112b.

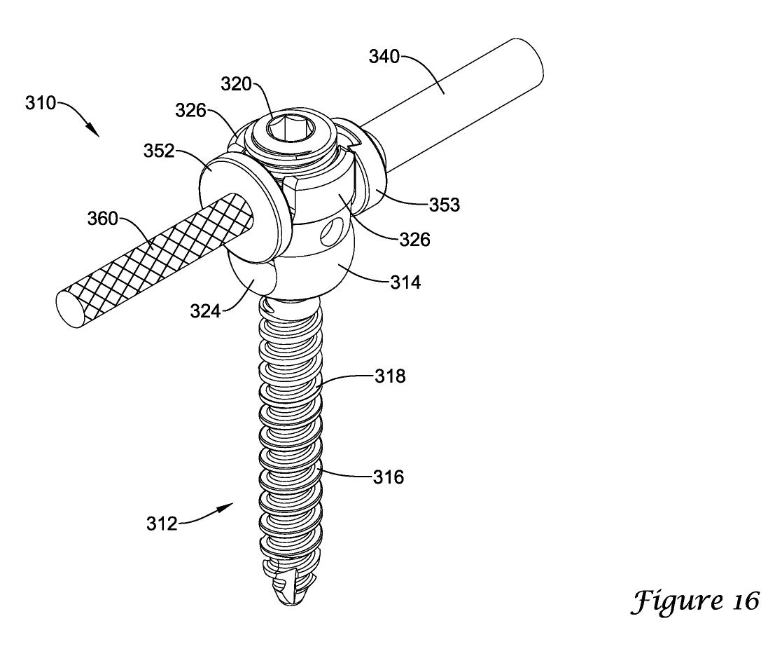

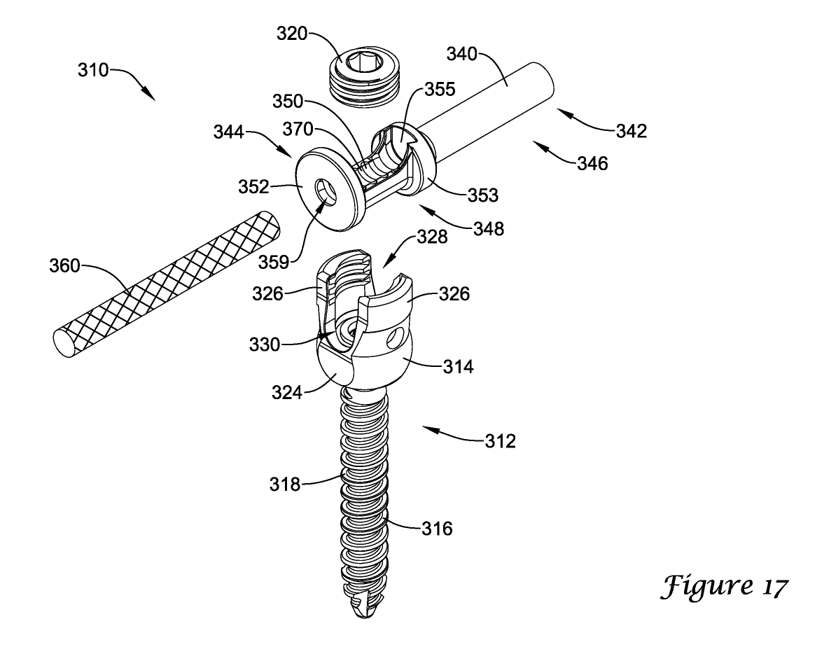

Another illustrative vertebral stabilization system 310 is illustrated at FIGS. 16 and 17. The vertebral stabilization system 310 may include one or more or a plurality of vertebral anchors or fasteners 312, one of which is shown in FIGS. 16 and 17. Although the vertebral anchors 312 are depicted as threaded vertebral fasteners (e.g., pedicle screws, bone screws), in some embodiments the vertebral anchors 312 may be vertebral hooks (e.g., laminar hooks) or other types of fastening members for attachment to a bony structure such as a vertebra of the spinal column. Each of the vertebral anchors 312 may be configured to be secured to a vertebra of a spinal column. For instance, the vertebral anchor 312 shown may be secured to a first vertebra, while a second vertebral anchor may be secured to a second vertebra, and a third vertebral anchor may be secured to a third vertebra, as described above regarding the vertebral stabilization system 110.

The vertebral anchor 312 may include a head portion 314 and a bone engagement portion 316 extending from the head portion 314. In some embodiments, the bone engagement portion 316 may be a shaft portion 318 of the vertebral anchor 312 extending from the head portion 314 along a longitudinal axis of the vertebral anchor 312. In some embodiments, the vertebral anchor 312 may be a monoaxial screw in which the head portion 314 is stationary relative to the shaft portion 318, and in other embodiments the vertebral anchor 312 may be a polyaxial screw in which the head portion 314 is actuatable (e.g., pivotable) relative to the shaft portion 318. In some embodiments, the shaft portion 318 may be configured to be installed into a bony region of a vertebra of the spinal column. For example, the shaft portion 318 may be installed into a pedicle of a vertebra, or other region of a vertebra. In some embodiments, the shaft portion 318 may be a threaded region having helical threads configured to be screwed into a pedicle of a vertebra, or other bony region of a vertebra.

The head portion 314 may include a base portion 324, from which the shaft portion 318 extends from, and first and second legs 326 extending from the base portion 324 on opposing sides of the head portion 314. The first and second legs 326 may define an opening 328, which may be a threaded opening in some instances, extending into the head portion 314 from an upper extent of the head portion 314 opposite the base portion 324. In embodiments in which the opening 328 is threaded, each of the first and second legs 326 may include a threaded portion for threadedly engaging a threaded portion of a securing member 320. In other embodiments, the first and second legs 326 may include other engagement features for engaging with a securing member positioned in the opening 328 between the first and second legs 326. The head portion 314 may additionally include a channel 330, such as a U-shaped channel, defined between the first and second legs 326. The channel 330 may extend through the head portion 314 from a first side of the head portion 314 to a second side of the head portion 314. The opening 328 may intersect the channel 330.

The vertebral anchor 312 may include a securing element, such as a threaded fastener 320 (e.g., a set screw, cap) configured to engage the head portion 314 to secure one or more elongate members to the vertebral anchor 312. For example, the threaded fastener 320 may include threads which mate with threads formed in the head portion 314. In other embodiments, other securing members, having engagement features, may be used to secure one or more elongate members, such as an elongate rod or flexible member, in the head portion 314 of the vertebral anchor 312.

The vertebral stabilization system 310 may also include one or more, or a plurality of elongate connecting members extending between vertebral anchors 312 of the vertebral stabilization system 310. As an illustrative example, the vertebral stabilization system 310 shown in FIGS. 16 and 17 includes a first elongate member, shown as an elongate rod 340, secured to the vertebral anchor 312, and a second elongate member, shown as a flexible member 360 (e.g., a flexible cord), also secured to the vertebral anchor 312. The elongate rod 340 may extend from the vertebral anchor 312 in a first direction to a second vertebral anchor while the flexible member 360 may extend from the vertebral anchor 312 in a second direction opposite the first direction to a third vertebral anchor.

As further illustrated in FIG. 17, the elongate rod 340 may have a first end 342, a second end 344, and a length between the first end 342 and the second end 344 sufficient to span the distance between the vertebral anchor 312 and a second vertebral anchor. The elongate rod 340 may be formed of any desired material, including those materials listed above such as stainless steel, commercially pure (CP) titanium, alpha-beta titanium alloy (e.g., Ti-6Al-4V), beta titanium alloy (e.g., Ti-15Mo-5Zr), other metals or metal alloys, polyether ether ketone (PEEK), PEEK composites, or other polymer materials.

The elongate rod 340 may include a first region 346 and a second region 348. The first region 346 may have a circular cross-section having a desired diameter, such as a diameter of about 5.5 millimeters, about 5.0 millimeters, about 4.5 millimeters, about 4.25 millimeters, about 4.0 millimeters, about 3.75 millimeters, or about 3.5 millimeters, in some instances. It is contemplated that the first region 346 may also have a non-circular cross-section in some instances.

In some instances, the second region 348 of the elongate rod 340 may be of a reduced diameter relative to the first region 346. For example, in some instances, the first region 346 may have a diameter of about 5.5 millimeters or more, while the second region 348 may have a diameter of less than 5.5 millimeters, such as about 5.0 millimeters, about 4.5 millimeters, about 4.25 millimeters, about 4.0 millimeters, about 3.75 millimeters, or about 3.5 millimeters. A transition region, such as a tapered region, or a step-wise transition may be located between the first region 346 and the second region 348.

The second region 348 may include at least a portion having an exterior engagement surface 350 against which the flexible member 360 may be positioned adjacent to. In some instances the exterior engagement surface 350, which is a portion of the exterior surface of the elongate rod 340, may be a planar surface. In other instances, the exterior engagement surface 350 may be a slightly convexly curved surface having a radius of curvature different from the radius of curvature of the remainder of the outer surface of the second region 348 of the elongate rod 340. For instance, the radius of curvature of the exterior engagement surface 350 may be greater than the radius of curvature of the outer surface around the circumference of the remainder of the second region 348. Thus, the center of curvature of the exterior engagement surface 350 may be offset from the central longitudinal axis of the elongate rod 340. In other embodiments, as shown in FIG. 17, the exterior engagement surface 350 may be a concave surface on the exterior of the elongate rod 340 forming an open channel along at least a portion of the second region 348 of the elongate rod 340 for placement of a portion of the flexible member 360 there along. Thus, the second region 348 may have a non-circular cross-section throughout at least a portion of the second region 348. The exterior engagement surface 350 may include surface roughenings 370. The surface roughenings 370 may help maintain the flexible member 360 from moving relative to the elongate rod 340 when the flexible member 460 and the elongate rod 340 are secured in the head portion 314 of the vertebral anchor 312. The surface roughenings 370 may be comprised of any mechanical gripping means such as, but not limited to, one or more threads, ribs, projections, grooves, teeth, and/or serrations or combination thereof.

The elongate rod 340 may also include a first flange 352 at the second end 344 of the elongate rod 340. In some instances, the first flange 352 may be generally circular with a center point coaxial with the central longitudinal axis of the elongate rod 340, while in other instances, the center point of the first flange 352 may be off-set from and non-coaxial with the central longitudinal axis of the elongate rod 340. The first flange 352 may include an opening 359 extending through the first flange 352.

The elongate rod 340 may also include a second flange 353 spaced away from the first flange 352 toward the first end 342 of the elongate rod 340. In some instances, the second flange 353 may be generally circular with a center point coaxial with the central longitudinal axis of the elongate rod 340, while in other instances, the center point of the second flange 353 may be off-set from and non-coaxial with the central longitudinal axis of the elongate rod 340. The second flange 354 may include a holding slot 355 for receiving the end portion of the flexible member 360. In some instances the holding slot 355 may have a trapezoidal shape or other shape such that a width of the slot nearer the first flange 352 is less than a width of the slot further from the first flange 352. In such an embodiment, an end portion of the flexible member 360 may be sized to have a cross-sectional dimension greater than a width of the slot 355 such that the end portion of the flexible member 360 may be urged into the slot 355 (e.g., in a direction perpendicular to the central longitudinal axis of the elongate rod 340) and retained in place by the interference fit between the end portion of the flexible member 360 and the side walls of the slot 355 such that the flexible member 360 may not be able to be readily removed from the slot 355 by pulling the flexible member 360 in a direction parallel to the central longitudinal axis of the elongate rod 340.

In some instances, the flexible member 360 may be pre-assembled with the elongate rod 340 prior to the medical procedure. For instance, the flexible member 460 may be positioned through the opening 359 of the first flange 352 and into the open channel formed by the concave exterior surface of the exterior engagement surface 350 prior to the medical procedure. In some instances, the flexible member 360 may be crimped in the open channel by crimping the portion of the elongate rod 340 between the first and second flanges 352, 353 which partially surround the flexible member 360 to provisionally secure the flexible member 360 to the elongate rod 340 prior to the medical procedure. In some instances, the concave exterior surface of the exterior engagement surface 350, while surrounding less than the entire circumference of the flexible member 360, may surround and contact greater than 50% of the circumference of the flexible member 360. Additionally and/or alternatively, the end portion of the flexible member 360 may be retained by the interference fit with the slot 355 to provisionally secure the flexible member 360 to the elongate rod 340 prior to the medical procedure.

The second flange 353 may be spaced from the first flange 352 such that when the elongate rod 340 is coupled to the vertebral anchor 312, the head portion 314 of the vertebral anchor 312 is positioned between the first and second flanges 352, 353 with the first flange 352 positioned adjacent a first side of the head portion 314 and the second flange 353 positioned adjacent a second side of the head portion 314.

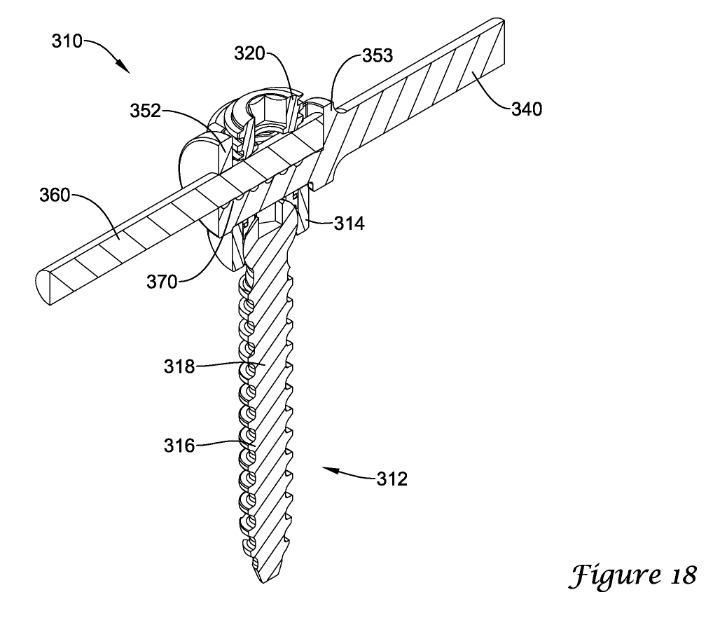

The flexible member 360, which in some instances may be a flexible cord, may be positioned adjacent to the elongate rod 340 in a side-by-side fashion in the head portion 314 of the vertebral anchor 312, with a portion of the flexible member 360 overlapping a portion of the elongate rod 340 such that an exterior surface of the flexible member 360 is positioned adjacent to and in contact with an exterior surface of the elongate rod 340. For example, a portion of the flexible member 360 may extend along and contact the exterior engagement surface 350 of the elongate rod 340. With such a configuration, the central longitudinal axis of the elongate rod 340 may be offset from and non-coaxial with the central longitudinal axis of the flexible member 360. FIG. 18 is a longitudinal cross-sectional view of the vertebral stabilization system 310 shown in FIG. 16 which further illustrates the overlapping positioning of the elongate rod 340 and the flexible member 360 in the head portion 314 of the vertebral anchor 312.

The elongate rod 340 may be positioned in the channel 330 of the head portion 314 of the vertebral anchor 312 such that the head portion 314 of the vertebral anchor 312 is positioned between the first and second flanges 352, 353 with the elongate rod 340 extending from the head portion 314 of the vertebral anchor 312 to the head portion of a second vertebral anchor (not shown). Thus, at least the portion of the second region 348 of the elongate rod 340 including the exterior engagement surface 350 may be positioned within the channel 330 of the head portion 314 of the vertebral anchor 312. Thus, a portion of the flexible member 360 overlapping and positioned adjacent the exterior engagement surface 350 of the elongate rod 340 may also be positioned within the channel 330 of the head portion of the vertebral anchor 312. The flexible member 360 may extend from the head portion 314 through the opening 359 of the first flange 352 to a third vertebral anchor (not shown).

In some instances, a spacer (not shown) may be disposed on the flexible member 360 and be positioned between the first flange 352 and the head portion of the third vertebral anchor, as described above. For instance, a spacer, such as the spacer 162 of the vertebral stabilization system 110 shown above at FIG. 12, may be disposed around the flexible member 360 and have a first end abutting or otherwise in contact with the first flange 352 and a second end positioned proximate the head portion of a third vertebral anchor.

The threaded fastener 320, or other securing element, may be engaged (e.g., rotatably or threadably engaged) to the head portion 314 to exert a clamping force on the flexible member 360 and the elongate rod 340 to secure the flexible member 360 and the elongate rod 340 in the channel 330 of the head portion 314 of the vertebral anchor 312. In some instances, the threaded fastener 320 may include a retention feature, such as one or more protrusions which may project into and/or deform the flexible member 360 when the threaded fastener 320 is compressed against the flexible member 360.

As shown in FIG. 18, in some instances the flexible member 360 may be positioned above the elongate rod 340 such that the elongate rod 340 rests against the base portion 324 and the flexible member 360 is positioned between the elongate rod 340 and the threaded fastener 320. Thus, in such instances the threaded fastener 320 may exert a force against the flexible member 360, which in turn exerts a force against the elongate rod 340 to secure the flexible member 360 and the elongate rod 340 in the channel 330 of the head portion 314 of the vertebral anchor 312. Positioning the flexible member 360 between the threaded fastener 320 and the elongate rod 340 may protect the elongate rod 340 from a notching effect (e.g., galling/fretting) attributed to direct contact between the threaded fastener 320 and the elongate rod 340 which may in turn increase the fatigue strength of the elongate rod 340. In other instances, the elongate rod 340 may be positioned above the flexible member 360 such that the flexible member 360 rests against the base portion 124 and the elongate rod 340 is positioned between the flexible member 360 and the threaded fastener 320. Thus, in such instances the threaded fastener 320 may exert a force against the elongated rod 340, which in turn exerts a force against the flexible member 360 to secure the flexible member 360 and the elongate rod 340 in the channel 330 of the head portion 314 of the vertebral anchor 312.

Those skilled in the art will recognize that the present invention may be manifested in a variety of forms other than the specific embodiments described and contemplated herein. Accordingly, departure in form and detail may be made without departing from the scope and spirit of the present invention as described in the appended claims.

What is claimed is:

1. A vertebral stabilization system comprising:
an elongate rod formed of a material having a modulus of elasticity less than or equal to 110 GPa and an ultimate strength greater than 1 GPa; and
a vertebral anchor for securement to a vertebra, the vertebral anchor including a head portion for receiving a portion of the rod.

2. The vertebral stabilization system of claim 1, wherein the elongate rod has a structural bending stiffness in the range of about 500,000 N-mm² to about 2,000,000 N-mm².

3. The vertebral stabilization system of claim 1, wherein the elongate rod has a structural bending stiffness of about 1,250,000 N-mm².

4. The vertebral stabilization system of claim 1, wherein the material is a beta titanium alloy.

5. The vertebral stabilization system of claim 1, wherein the material is high strength Ti-15Mo-5Zr.

6. The vertebral stabilization system of claim 1, wherein the elongate rod has a diameter in the range of about 3.25 millimeters to about 4.5 millimeters.

7. The vertebral stabilization system of claim 2, wherein the material is high strength Ti-15Mo-5Zr.

8. The vertebral stabilization system of claim 7, wherein the elongate rod has a diameter in the range of about 3.25 millimeters to about 4.5 millimeters.

9. The vertebral stabilization system of claim 1, wherein the elongate rod has a fatigue strength greater than that of a 5.5 millimeter rod formed of commercially pure (CP) titanium.

10. The vertebral stabilization system of claim 1, wherein the head portion of the vertebral anchor includes a channel extending therethrough for receiving a portion of the rod, the channel having a diameter sized to receive a rod having a diameter of 5.5 millimeters, wherein the rod has a diameter of 4.5 millimeters or less.

11. A vertebral stabilization system for a spinal column, the system comprising:
a vertebral anchor for securement to a vertebra, the vertebral anchor including a head portion having first and second arms extending from a base of the head portion, the head portion including a channel defined between the first and second arms extending between a first side and a second side of the head portion;
an elongate rod having a first region and a second region, the first region of the elongate rod including an outer surface having an engagement surface portion;
a flexible member having a first region and a second region, the first region of the flexible member positionable adjacent the engagement surface portion of the first region of the elongate rod when the first region of the elongate rod and the first region of the flexible member are received in the channel of the head portion of the vertebral anchor; and
a securing member configured to engage the first and second arms of the head portion of the vertebral anchor to secure both the elongate rod and the flexible member in the channel of the head portion of the vertebral anchor.

12. The vertebral stabilization system of claim 11, wherein the securing member presses the first region of the flexible member against the engagement surface portion of the first region of the elongate rod when secured in the channel of the head portion of the vertebral anchor.

13. The vertebral stabilization system of claim 12, wherein the elongate rod extends from the first side of the head portion of the vertebral anchor and the flexible member extends from the second side of the head portion of the vertebral anchor.

14. The vertebral stabilization system of claim 13, wherein the elongate rod includes a flange positioned adjacent the second side of the head portion of the vertebral anchor, the flange including a notch or opening for receiving the flexible member therethrough.

15. The vertebral stabilization system of claim 14, further comprising:
a second vertebral anchor for securement to a second vertebra, the second vertebral anchor including a head portion; and
a spacer having a first end, a second end and a lumen extending from the first end to the second end for receiving the flexible member therethrough;
wherein the first end of the spacer is positionable between the flange of the elongate rod and the head portion of the second vertebral anchor when the flexible member is secured in the head portion of the second vertebral anchor.

16. The vertebral stabilization system of claim 11, wherein the first region of the elongate rod has a first diameter and the second region of the elongate rod has a second diameter greater than the first diameter.

17. A method of stabilizing the spinal column of a patient, comprising:
securing first and second vertebral anchors to first and second vertebrae of the spinal column on a first, lateral side of the spinal column;
securing third and fourth vertebral anchors to the first and second vertebrae of the spinal column on a second, contra-lateral side of the spinal column;
securing a first elongate rod to the first and second vertebral anchors on the first, lateral side of the spinal column;
securing a second elongate rod to the third and fourth vertebral anchors on the second, contra-lateral side of the spinal column; and
transferring a spinal load between the first and second vertebrae, the first and second vertebrae including anterior elements and posterior elements, wherein between 17% to 19% of the spinal load is transferred through the posterior elements.

18. The method of claim 17, wherein the first and second vertebrae are located in a lumbar region of the spinal column.

19. The method of claim 17, wherein about 18% of the spinal load is transferred through the posterior elements.

20. The method of claim 17, wherein each of the first and second elongate rods is formed of a material having a modulus of elasticity less than or equal to 110 GPa and an ultimate strength greater than 1 GPa.

21. The method of claim 17, wherein each of the first and second elongate rods has a structural bending stiffness in the range of about 500,000 N-mm² to about 2,000,000 N-mm².

22. The method of claim 17, wherein each of the first and second elongate rods is formed of high strength Ti-15Mo-5Zr and has a diameter in the range of about 3.25 millimeters to about 4.5 millimeters.

23. A method of stabilizing a lumbar region of a spinal column, the method comprising:
installing a first vertebral anchor on a first lumbar vertebra;
installing a second vertebral anchor on a second lumbar vertebra; and
securing an elongate rod between the first vertebral anchor and the second vertebral anchor, the elongate rod having a diameter of less than 5.5 millimeters.

24. The method of claim 23, wherein the elongate rod is formed of a material having a modulus of elasticity less than or equal to 110 GPa and an ultimate strength greater than 1 GPa.

25. The method of claim 23, wherein the elongate rod is formed of high strength Ti-15Mo-5Zr and has a diameter in the range of about 3.25 millimeters to about 4.5 millimeters.

26. The method of claim 23, wherein the elongate rod has a structural bending stiffness in the range of about 500,000 N-mm² to about 2,000,000 N-mm².

27. A vertebral stabilization system, comprising:
an elongate rod having a diameter of 4.5 millimeters or less;
a vertebral anchor for securement to a vertebra, the vertebral anchor including a head portion having a first leg, a second leg and a channel extending between the first leg and the second leg for receiving the elongate rod; and
a securing member configured for securement of the elongate rod in the channel of the head portion of the vertebral anchor, the securing member including a first component rotatably coupled to a second component, the first component configured for engagement with the first and second legs of the head portion of the vertebral anchor and the second component configured for engagement with the elongate rod;
wherein the elongate rod secured to the head portion of the vertebral anchor with the securing member has a fatigue strength greater than a spinal rod of a diameter of 5.5 millimeters formed of any of stainless steel, commercially pure (CP) titanium, Ti-6Al-4V alpha-beta titanium alloy, Ti-6Al-7Nb alpha-beta titanium alloy, or cobalt-chromium-molybdenum alloy (Co—Cr—Mo) secured to a bone screw with a set screw in direct contact with the spinal rod.

28. The vertebral stabilization system of claim 27, wherein the first component is formed of a first material having a modulus of elasticity and the second component is formed of a second material having a modulus of elasticity less than the modulus of elasticity of the first material.

29. The vertebral stabilization system of claim 28, wherein the first material is a metallic material and the second material is a polymeric material.

30. The vertebral stabilization system of claim 28, wherein the elongate rod is formed of a third material having a modulus of elasticity, wherein the modulus of elasticity of the second material is less than the modulus of elasticity of the third material.

31. The vertebral stabilization system of claim 27, wherein the second component includes a boss which extends into an opening of the first component to rotatably couple the first and second components together.

32. The vertebral stabilization system of claim 27, wherein the channel of the head portion of the vertebral anchor is sized to receive an elongate rod having a diameter of 5.5 millimeters or more.

33. The vertebral stabilization system of claim 27, wherein the elongate rod is formed of a material having a modulus of elasticity less than or equal to 110 GPa and an ultimate strength greater than 1 GPa.
